SEQUENCE LISTING (2 sequences)

SEQ ID NO: 1 - RNA, 23 nt, Artificial Sequence
  sgRNA sequence ′targeting GFP′
gmgmcmcacaaguucagcguguc

SEQ ID NO: 2 - RNA, 23 nt, Artificial Sequence
  sgRNA sequence ′non-targeting′
cmgmumacgaucucguaaacgcg

(19) United States
(12) Patent Application Publication
(10) Pub. No.: US 2024/0173717 A1
(43) Pub. Date: May 30, 2024

(54) MULTISTAGE DEVICE AND METHOD FOR INTRACELLULAR DELIVERY

(71) Applicants: Max-Planck-Gesellschaft zur Förderung der Wissenschaften e.V., München (DE); Technische Universität Dresden, Dresden (DE)

(72) Inventors: Jochen GUCK, Leipzig (DE); Salvatore GIRARDO, Erlangen (DE); Ruchi GOSWAMI, Erlangen (DE); Shada HOFMEIER ABU HATTUM, Erlangen (DE); Jörg MANSFELD, Achim (DE); Alena UVIZL, Chocomysl (CZ)

(21) Appl. No.: 18/284,798

(22) PCT Filed: Mar. 30, 2022
(86) PCT No.: PCT/EP2022/058435

§ 371 Date: Sep. 28, 2023

(30) Foreign Application Priority Data
Apr. 1, 2021 (EP) 21166760.5

(51) Int. Cl. B01L 3/00

(52) U.S. Cl. CPC B01L 3/502761; B01L 2200/027; B01L 2300/047; B01L 2300/0663; B01L 2300/0681; B01L 2400/0487; B01L 2400/082

(57) ABSTRACT

The present invention relates to a microfluidic device for introducing pores into and/or enhancing the diameter of pores in the cell membrane of a cell by cell deformation for delivery of cargo molecules into said cell, the device comprising: an inlet and an outlet; and at least one microfluidic channel positioned between said inlet and said outlet, defining a lumen, adapted to allow a cell and cargo molecules in a suspension solution to pass therethrough; wherein the at least one microfluidic channel comprises at least two constrictions with different cross-sections, wherein one of said constrictions has a cross-section that is larger than the average cross-section of said cell and adapted to apply hydrodynamic forces to said cell and a second of said constrictions has a cross-section that is equal to or smaller than the average cross-section of said cell and adapted to apply contact-based compression forces to said cell, while allowing said cell to pass through said constrictions.

The field of the invention relates to microfluidic systems and more specifically microfluidic systems that are used for delivery of materials into cells while preserving cell viability and cell functionality.

The present invention relates to a microfluidic device for introducing pores into and/or enhancing the diameter of pores in the cell membrane of a cell by cell deformation for delivery of cargo molecules into said cell, the device comprising: an inlet and an outlet; and at least one microfluidic channel positioned between said inlet and said outlet, defining a lumen, adapted to allow a cell and cargo molecules in a suspension solution to pass therethrough; wherein the at least one microfluidic channel comprises at least two constrictions with different cross-sections, wherein one of said constrictions has a cross-section that is larger than the average cross-section of said cell and adapted to apply hydrodynamic forces to said cell and a second of said constrictions has a cross-section that is equal to or smaller than the average cross-section of said cell and adapted to apply contact-based compression forces to said cell, while allowing said cell to pass through said constrictions.

In this specification, a number of documents are cited. The disclosure of these documents, while not considered relevant for the patentability of this invention is herewith incorporated by reference in its entirety. More specifically, all referenced documents are incorporated by reference to the same extent as if each individual document was specifically and individually indicated to be incorporated by reference.

Delivery of cargo molecules such as proteins, drugs or nucleic acids, into cells is crucial for various biological and pharmaceutical applications such as cell transfection, drug delivery or gene editing. Intracellular delivery faces several challenges, most importantly, membrane-impermeable cargos and hard-to-transfect cells due to the heterogeneity of biological samples. Conventional methods for intracellular delivery use electrical, chemical or mechanical manipulations to permeabilize the cell membrane for allowing entrance of the cargo to the cytoplasm.

Mechanoporation i.e. mechanical manipulation relies on mechanical forces to deform cells in suspension allowing for transient plasma membrane permeabilization in a high-throughput, well-controlled and reproducible manner. Recently developed microfluidic mechanoporation techniques even provided access to hard-to-transfect cells by transiently creating pores in cell membranes by means of shear-induced and constriction contact-based rapid cell deformation. Since these techniques do not require further modifications of molecules to be delivered, any external electric field effects or use of viral vectors, all related biological toxicities and other limitations of biochemical methods can be overcome¹⁻³.

A pioneering study employed a syringe loading technique to transiently permeabilize the cell membrane by directly applying the shear forces through hypodermic needle⁴. Hallow et al. demonstrated that by simply flowing cells through microchannels, shear-induced plasma membrane disruption leads to intracellular uptake of the biomolecules. They found that the best delivery efficiency was obtained by applying high flow rates to small-diameter, short-length channels, suggesting that exposure to high shear stress for a short duration of time led to the most optimal results. Even though shear-induced intracellular delivery approaches are straightforward, they lack high loading efficiency and optimal cell viability⁵.

Higher delivery efficiency of different cargos in different cells, while keeping good cell viability has been achieved by using solid contact-mediated membrane permeabilization by fast squeezing of cells in suspension through constrictions⁶⁻¹³. Sharei et al. employed silicon-based microfluidic devices operated by an external pressure regulator to control the shear and compression rates experienced by cells for cytosolic diffusive delivery (see also US 2019/093073). This delivery method was dependent on cargo size due to size-dependent diffusive molecules loading and different cell types under the same treatment showed different behavior in uptaking the same or different cargos⁶. Lam et al. enlarged the applicability of the same method to PDMS-based device (Cyto-PDMS) controlled by a syringe pump, making the device fabrication low-cost and accessible to rapid prototyping for the easy optimization in most academic research labs¹⁴. However, their method required a multi-casting PDMS process and the use of syringe pump-driven flow required longer stabilization time compared to pressure-driven flow in microfluidic systems¹⁵, precluding the stable and reproducible processing of small sample volumes. Furthermore, a significant decline in cell viability appeared for high flow rates and heterogeneous delivery, cargo and cell-size dependent, was observed.

To enlarge cytosolic delivery by mechanoporation to a broad range of cargo molecules, Liu et al. recently presented a novel method with molecular size-independent delivery efficiency, showing the importance of timescale deformation and cell mechanical properties in mechanoporation^{16,17}. The molecules were loaded into cells by a phenomenon called cell volume exchange for convective transfer (cell VECT), implemented in a microfluidic system by rapidly passing cells through a microchannel in which they undergo sudden deformations under several ridges. Delivery mostly into human cells (K562) of different cargos with high efficiency regardless of molecule size and without impairing cell viability was shown. Even if the molecular loading was independent of molecules' size, it was dependent on cell size, velocity and a threshold number of ridges and gap size were needed to obtain high delivery efficiency. Their analysis was mostly focused on compliant cells (K562) and a further study showed that VECT was dependent on timescale deformation (compression time) and cell mechanical properties, suggesting that the efficiency of the method might vary among different cells. Following the principle of volume exchange during fast deformation Kizer et al. introduced a new method called hydroporator, based on the rapid hydrodynamic cell shearing inside a microfluidic device under high Reynolds number (Re>10²). Increasing the flow rate increased delivery efficiency, but cell viability decreased. Furthermore, the delivery efficiency was dependent on cell deformability and cargo size and the delivery of different cargos was shown mostly on compliant cells¹⁸.

FIGS. 1 and 2 show existing methods of mechanoporation relying either on high shear forces or contact-based deformation. High shear forces are achieved by these systems due to high Reynolds numbers (Re>10²). Increasing the flow rate in these methods will lead to an increase in the cargo delivery efficiency, however cell viability is decreased. In contact-based deformation of cells, efficiency of delivery can be improved, without increasing the flow rate, by constricting the cells in a narrower constriction than the cell diameter. This, however, leads to varying results when comparing different cell types due to the heterogeneity of cells sizes and mechanical properties. Increasing the number of contact-based constrictions will improve the discrepancy in delivery for different cell types, however, it will lead to a decrease in viability and throughput (cells slowdown in every constriction) of transfected cells.

Thus, existing methods are highly specific to cargo size or cells properties and result in decreased efficiency, throughput and viability of treated cells. Accordingly, there is still an urgent need in the art to provide means and methods for intracellular delivery which can overcome deficiencies of conventional techniques. The present invention addresses this need and provides devices, systems and methods for highly efficient and improved intracellular delivery based on progressive mechanoporation.

Accordingly, the present invention relates in a first aspect to a microfluidic device for introducing pores into and/or enhancing the diameter of pores in the cell membrane of a cell by cell deformation for delivery of cargo molecules into said cell, the device comprising: an inlet and an outlet; and at least one microfluidic channel positioned between said inlet and said outlet, defining a lumen, adapted to allow a cell and cargo molecules in a suspension solution to pass therethrough; wherein the at least one microfluidic channel comprises at least two constrictions with different cross-sections, wherein one of said constrictions has a cross-section that is larger than the average cross-section of said cell and adapted to apply hydrodynamic forces to said cell and a second of said constrictions has a cross-section that is equal to or smaller than the average cross-section of said cell and adapted to apply contact-based compression forces to said cell, while allowing said cell to pass through said constrictions.

Generally, a “microfluidic device” is a device that enables the processing of a fluid on a very small scale, typically measured in volumes such as milliliter (mL), microliter (μL), nanoliter (nL), picoliter (pL), or femtoliter (fL) and/or by physical scale such as millimeter (mm), micrometer (μm) (also referred to as “micron”), nanometer (nm), etc.

The term “microfluidic device” as used herein broadly encompasses a device having one or more channels through which moving fluid is directed. The term “fluid” as used herein refers to a liquid, e.g. cell suspension solution containing cargo molecules. Accordingly, as used herein, the term “channel” refers to a fluid flow pathway. The terms “inlet” and “outlet” refer to a terminal opening wherein a fluid enters or exits the microfluidic device or the microfluidic channel, respectively. The term “lumen” as used herein refers to the volume which is enclosed by the microfluidic channel. The term “constriction” as used herein refers to a reduction of the original cross-sectional area of the microfluidic channel. A “cross-section” as used herein refers to the orthogonal section to the longitudinal axis of the channel.

The microfluidic device of the invention facilitates delivery of cargo molecules (such as therapeutically relevant molecules) into a cell. The term “cell” as used herein relates to eukaryotic cells or prokaryotic cells. The term “eukaryotic cell” as used herein refers to any cell of a eukaryotic organism. The eukaryotic cell preferably is a mammalian cell. The term “mammalian cell” as used herein, is well known in the art and refers to any cell belonging to an animal that is grouped into the class of mammalia. Typical mammalian cells include, for example, HeLa, RPE-1, U2OS cells, planarian stem cells (neoblasts), immune cells (T cells, B cells, neutrophils), stem cells (mesenchymal) and other cell lines well-known in the art.

The term “prokaryotic cell” as used herein refers to any cell of a prokaryotic organism. Representative examples of prokaryotic cells are E. coli, Streptomyces and Salmonella typhimurium cells.

The microfluidic device of the invention may also facilitate delivery of cargo molecules into spheroplasts. The term “spheroplast” as used herein refers to a cell from which the cell wall has been almost completely removed. Methods for producing spheroplasts are well-known in the art.

The term “diameter” as used herein refers to the maximal length of a straight line passing through the center of a described element. The term “diameter”, as used herein, encompasses diameters of spherical elements as well as of non-spherical elements.

A “suspension solution” as used herein is any physiologic or cell-compatible buffer or solution comprising cells and cargo molecules. For example, a suspension solution can be a cell nutrition medium. The cell nutrition medium can be a culturing or a growth medium and the exact ingredients will depend on the type of cells the cell suspension contains. An example for a cell nutrition medium containing Hela cells as used herein is Dulbecco's Modified Eagle's Medium (DMEM, Gibco, Cat. no. 41966), supplemented with 10% (v/v) FBS (Gibco), 1% (v/v) Glutamax (Gibco), 1% (v/v) penicillin-streptomycin (Sigma-Aldrich) and 0.5 μg/ml amphotericin B (Sigma Aldrich).

A “cargo molecule” as used herein refers to an entity to be delivered inside a cell. Cargo molecules include, but are not limited to small molecules, amino acids, proteins, nucleic acids, impermeable dyes, nano-carrier systems, CRISPR/Cas9 gene-editing complexes and combinations thereof.

As can be taken from Example 6 herein below, progressive membrane mechanoporation was also used to efficiently deliver cargo molecules with a molecular weight of 190.8 kDa into cells. Hence, in accordance with the present invention, the molecular weight of the cargo molecules is preferably ≤200 kDa. However, progressive cell membrane mechanoporation as described herein may be used to deliver cargo molecules with a molecular weight >200 kDa into cells with high delivery efficiency while maintaining high cell viability by adapting the cell velocity, the viscosity of the medium and/or the channel geometry, including the arrangement and dimensions of the constrictions.

The term “small molecule” generally relates to molecules having low molecular weight. The “small molecule” as used herein may be an organic molecule. Organic molecules relate or belong to the class of chemical compounds having a carbon basis, the carbon atoms linked together by carbon-carbon bonds. The original definition of the term organic related to the source of chemical compounds, with organic compounds being those carbon-containing compounds obtained from plant or animal or microbial sources, whereas inorganic compounds were obtained from mineral sources. Organic compounds can be natural or synthetic. The organic molecule is preferably an aromatic molecule and more preferably a heteroaromatic molecule. In organic chemistry, the term aromaticity is used to describe a cyclic (ring-shaped), planar (flat) molecule with a ring of resonance bonds that exhibits more stability than other geometric or connective arrangements with the same set of atoms. Aromatic molecules are very stable, and do not break apart easily to react with other substances. In a heteroaromatic molecule at least one of the atoms in the aromatic ring is an atom other than carbon, e.g. N, S, or O.

Alternatively, the “small molecule” in accordance with the present invention may be an inorganic compound. Inorganic compounds are derived from mineral sources and include all compounds without carbon atoms (except carbon dioxide, carbon monoxide and carbonates).

Typically, a small molecule has a molecular weight of less than about 2000 Da. The size of a small molecule can be determined by methods well-known in the art, e.g., mass spectrometry.

The term “protein” as used herein is used interchangeably with the term (poly)peptide and refers to polymers constructed from one or more chains of amino acid residues linked by peptide bonds. (Poly)peptides describe a group of molecules which comprise the group of peptides consisting of up to 30 amino acids, as well as the group of polypeptides consisting of more than 30 amino acids. The term “protein” also refers to chemically or post-translationally modified proteins. The terms apply to naturally occurring amino acid polymers, as well as to amino acid polymers in which one or more amino acid residue is an artificial chemical analogue of a corresponding naturally occurring amino acid. For example, proteins as contemplated herein include, but are not limited to bioactive proteins, such as transcription factors, enzymes, antibodies, diagnostic proteins and therapeutic proteins.

As used herein, the term “nucleic acid” refers to deoxyribonucleotides or ribonucleotides, as well as to deoxyribonucleotide or ribonucleotide polymers in either single- or double-stranded form, and unless otherwise limited, encompasses known analogues having the essential nature of natural nucleotides in that they hybridize to single-stranded nucleic acids in a manner similar to naturally occurring nucleotides. Included are nucleic acid mimicking molecules known in the art such as synthetic or semi-synthetic derivatives of DNA or RNA and mixed polymers. Such nucleic acid mimicking molecules or nucleic acid derivatives according to the invention include phosphorothioate nucleic acid, phosphoramidate nucleic acid, 2′-O-methoxyethyl ribonucleic acid, morpholino nucleic acid, hexitol nucleic acid (HNA), peptide nucleic acid (PNA) and locked nucleic acid (LNA) (see Braasch and Corey, Chem Biol 2001, 8: 1)³³. LNA is an RNA derivative in which the ribose ring is constrained by a methylene linkage between the 2′-oxygen and the 4′-carbon. Also included are nucleic acids containing modified bases, for example thio-uracil, thio-guanine and fluoro-uracil. A nucleic acid molecule typically carries genetic information, including the information used by cellular machinery to make (poly)peptides. Furthermore, nucleic acids as contemplated herein include, but are not limited to siRNAs, dsRNAs, miRNAs, antisense molecules or nucleic acid catalysts, such as ribozymes and aptamers.

The term “impermeable dye” as used herein generally relates to a dye that cannot diffuse through a cell membrane by passive diffusion either because it is e.g. too hydrophilic or too large. As used herein, the term “dye” refers to a molecule or part of a compound which absorbs specific frequencies of light.

The term “nano-carrier systems” as used to herein refers to colloidal drug carrier systems having submicron particle size, typically below 500 nm. For example, nano-carrier systems as contemplated herein include, but are not limited to nano rods and quantum dots.

As the cell in a suspension solution passes through a constriction of a microfluidic channel of the invention, it experiences deforming forces that result in cell deformation. “Cell deformation” as used herein refers to a transformation or change of the shape of a cell, wherein the degree of cell deformation depends on, amongst others, the cell size, cell mechanical properties, e.g. cell elasticity, and cell velocity. Cell deformation can be defined by the ratio between the major and the minor cell axis, wherein the ratio of a cell in the relaxed state is normalized to 1. Hence, the dimension/area of the cross-section of the constrictions can be customized to control the degree of cell deformation and subsequently also the delivery efficiency.

It is important to note that in the practical use of the device of the invention, cells experience progressive deformation, i.e. iterative deformation, based on the combination of at least two constrictions, wherein one of said constrictions is adapted to apply hydrodynamic forces to the cell and wherein a second of said constrictions is adapted to apply contact-based compression forces to the cell. The term “hydrodynamic forces” as used in accordance with the present invention relates to forces applied to the cell as a result of its movement/velocity and/or the flow of the surrounding medium. The hydrodynamic forces include shear forces and forces due to the pressure gradient originating from inertial and viscous forces. In this case, physical contact between the constriction and the cell is not required. The term “contact-based compression force” as used in accordance with the present invention relates to forces applied on the cell as a result of passing through a channel that is smaller or equal to the cell diameter. In this case, physical contact between the constriction and the cell is required. The hydrodynamic forces and the contact-based compression forces are controlled by changing the flow rate of the medium, the viscosity of the medium, the pressure along the channel and/or changing the channel geometry.

In particular, one of said constrictions is adapted to apply hydrodynamic forces to pre-deform the cell, where a pre-deformed cell can already exhibit pores in the membrane. Cell pre-deformation before the contact-based compression may be used to avoid mechanical shock on cells that approach narrow constrictions and to facilitate their transition reducing the impact on cell viability. This enlarges the range of applicable pressure/flow and compression not affecting cell viability with a consequent improvement in delivery efficiency among cells with different physical properties (e.g., cell size and mechanical properties). Pre-deformed cells can cross the microchannels length in shorter time, leading to shorter timescale deformation. Furthermore, pre-deforming cells with different elasticity homogenize the way cells travel in microchannels and provide comparable treatment among cells with different mechanical properties.

Accordingly, one of the at least two constrictions has a cross-section that is larger than the average cross-section of said cell and hence adapted to apply hydrodynamic forces to the cell and a second of said constrictions has a cross-section that is equal to or smaller than the average cross-section of said cell and hence adapted to apply contact-based compression forces to the cell, while allowing said cell to pass through said constrictions.

The fluid shear stress (FSS, τ) for rectangular channel with h>w was calculated as τ=6μQ/hw², where μ is the dynamic viscosity of the surrounding medium (N·s m⁻²), Q is the volumetric flow rate in the first constriction (m³ s⁻¹), h and w are the height and width of the first constriction, respectively. Contact-based compression is evaluated as a ratio between the size of the channel and the diameter of the cell.

Cell deformation as described herein above subsequently results in the introduction of pores into and/or the enhancement of the diameter of pores in the cell membrane of a cell. The term “cell membrane” as used herein refers to the lipid-containing barrier which separates cells or groups of cells from the extracellular space. “Pores” as used herein refer, without limitation, to openings, such as natural openings as well as temporary perturbations, not based on natural openings, in the cell membrane which allow and/or facilitate the delivery of cargo molecules from outside the cell to move into the cell. Hence, “pores” as used herein induce and/or enhance permeabilization of the cell membrane of a cell. Natural openings include but are not limited to transmembrane channels such as ion channels. Upon recovery of the cell membrane, a portion of the cargo molecules is then entrapped inside the cell. The pores obtained with the device of the invention have a diameter that allows and/or facilitates the delivery of cargo molecules from outside the cell to move into the cell. For example, pores may have a diameter between 2 nm and 100 nm.

Generally, delivery efficiency of cargo molecules into the cell(s) depends on various factors, including cell and cargo molecule size as well as cell elasticity.

Larger cells generally experience a higher degree of strain induced by hydrodynamic forces and contact-based compression forces when passing through the constrictions of the device of the invention, causing a higher degree of deformation. However, abrupt and strong cell deformation as used in previous mechanoporation techniques can significantly reduce cell viability and proliferation capacity.

Furthermore, delivery efficiency also depends on the mechanical properties, i.e. elasticity, of the cell. Conventionally, cell elasticity is defined by the Young's Modulus, wherein a higher modulus corresponds to stiffer cells. At the same ratio of cell diameter to constriction width, delivery is generally thought to be more efficient into softer than into stiffer cells (see also FIG. 10a). Surprisingly, however, in accordance with the present invention it was found that the microfluidic device of the invention can be used to efficiently deliver small cargos into cells independent of the cell elasticity (cf. Example 4 as described herein below).

This is because progressive mechanoporation, in contrast to the abrupt cell deformation as previously used, can avoid mechanical shock on cells. Moreover, it improves delivery efficiency across different cell types through molecular loading aided by the combination of convective and diffusive transport.

The examples herein below describe the application of different microfluidic devices in agreement with the first aspect of the invention, wherein the Reynolds number (Re), the fluid shear stress (τ) and the average cell velocity as well as the applied pressures were ranging from 6.9-19.6, 149.5-428.5 N m⁻² and 391-1047 mm s⁻¹, respectively (Table 1). Moreover, delivery of different cargo sizes, including cargo with a molecular weight of 4000 Da, 7000 Da and 190000 Da in size has been analyzed.

As generally intracellular delivery is not only dependent on the cargo size but also on the complexity of the cargo in terms of structure and composition, delivery efficiency of protein complexes greatly varies with regard to their chemical properties. Therefore, there is currently no universal method for protein delivery, as in the case of plasmids¹⁹. Importantly, delivery of Cas9-sgRNA in the form of ribonucleoproteins is preferred over delivery in the form of plasmids due to higher gene editing efficiency and minimal off-target effects. There is no fully established technique used as a gold standard for Cas9-sgRNA RNPs delivery²⁰⁻²². Even a very promising technique such as electroporation displays off-targets. Specifically, it has been shown that electroporation causes misexpression of 34% of all genes and unspecific upregulation of cytokines in human T cells⁹.

In summary, in accordance with the present invention it was found that, compared to the shear-induced and contact-mediated deformation methods, progressive cell membrane mechanoporation (PM), i.e. iterative cell deformation using a combination of hydrodynamic and contact-based compression forces provides high delivery efficiency among cells with different mechanical properties, enhanced by fast and gradual deformation under high hydrodynamic and compression forces in shorter and narrower constrictions. As can be taken from the examples herein below, it was found that such high delivery efficiency can be achieved without adverse effects on cell viability and proliferation capacity and even allows the delivery of large cargo molecules.

To the best knowledge of the inventors there is currently no other device having all these advantageous characteristics, thereby providing biomedical research and industry with a new device for highly efficient and improved intracellular delivery.

In accordance with a preferred embodiment of the first aspect of the invention, the ratio between the minimum dimension of the cross-section of the large constriction and the minimum dimension of the cross-section of the small constriction is between 1.2 and 7, preferably between 2.5 and 5, more preferably between 2.5 and 3.5; and/or the ratio between the area of the cross-section of the large constriction and the area of the cross-section of the small constriction is between 1.2 and 7, preferably between 2.5 and 5, more preferably between 2.5 and 3.5; and/or the ratio between the minimum dimension of the cross-section of the large constriction and the minimum dimension of the cross-section of the microfluidic channel between the large and small constrictions is between 1.1 and 3.5, preferably between 1.2 and 2.5, more preferably between 1.3 and 2.0; and/or the ratio between the area of the cross-section of the small constriction and the area of the cross-section of the microfluidic channel between the large and small constrictions is between 0.1 and 0.9, preferably between 0.2 and 0.6, more preferably between 0.2 and 0.4.

Accordingly, the geometry of the at least two constrictions of the invention can be customized to enhance delivery efficiency and cell viability into different cell types (e.g. cells of different size, different elasticity etc.). For example, the geometry of the microfluidic device as defined in the following preferred embodiment of the first aspect of the invention is preferably used for cells with an average diameter of 15 μm (e.g. RPE-1 cells with an average diameter of 14.3±2.2 μm and HeLa K cells with an average diameter of 14.7±2.3 μm; see Example 3 herein below), independent of cell elasticity.

In accordance with another preferred embodiment of the first aspect of the invention, the minimum dimension of the cross-section of the large constriction and/or the minimum dimension of the cross-section of the small constriction is between 2 μm and 20 μm, preferably between 2 μm and 15 μm, more preferably between 4 μm and 8 μm; and/or the area of the cross-section of the large constriction and/or the area of the cross-section of the small constriction is between 25 μm² and 1200 μm², preferably between 30 μm² and 900 μm², more preferably between 45 μm² and 250 μm²; and/or the large constriction and/or the small constriction extends over a length along the fluid flow direction between 10 μm and 50 mm, preferably between 10 μm and 15 mm, more preferably between 20 μm and 60 μm; and/or the distance between the large constriction and the small constriction along the fluid flow direction is between 50 μm and 5 mm, preferably between 50 μm and 1 mm, more preferably between 50 μm and 200 μm; and/or the minimum dimension of the cross-section of the microfluidic channel between the large and small constrictions is between 7 μm and 19 μm, preferably between 8 μm and 15 μm, more preferably between 8 μm and 10 μm; and/or the area of the cross-section of the microfluidic channel between the large and small constrictions is between 80 μm² and 1100 μm², preferably between 100 μm² and 600 μm², more preferably between 150 μm² and 450 μm².

In another preferred embodiment of the first aspect of the invention, the minimum dimension of the cross-section of the large constriction and/or the minimum dimension of the cross-section of the small constriction is between 2 μm and 20 μm; and/or the area of the cross-section of the large constriction and/or the area of the cross-section of the small constriction is between 25 μm² and 1200 μm²; and/or the large constriction and/or the small constriction extend over a length along the fluid flow direction between 10 μm and 50 mm; and/or the distance between the large constriction and the small constriction along the fluid flow direction is between 50 μm and 5 mm; and/or the minimum dimension of the cross-section of the microfluidic channel between the large and small constrictions is between 7 μm and 19 μm; and/or the area of the cross-section of the microfluidic channel between the large and small constrictions is between 80 μm² and 1100 μm²; and the ratio between the minimum dimension of the cross-section of the large constriction and the minimum dimension of the cross-section of the small constriction is between 1.2 and 7; and/or the ratio between the area of the cross-section of the large constriction and the area of the cross-section of the small constriction is between 1.2 and 7; and/or the ratio between the minimum dimension of the cross-section of the large constriction and the minimum dimension of the cross-section of the microfluidic channel between the large and small constrictions is between 1.1 and 3.5; and/or the ratio between the area of the cross-section of the small constriction and the area of the cross-section of the microfluidic channel between the large and small constrictions is between 0.1 and 0.9.

In a further preferred embodiment of the first aspect of the invention, the minimum dimension of the cross-section of the large constriction and the minimum dimension of the cross-section of the small constriction is between 2 μm and 20 μm; and/or the area of the cross-section of the large constriction and/or the area of the cross-section of the small constriction is between 25 μm² and 1200 μm²; and/or the large constriction and/or the small constriction extend over a length along the fluid flow direction between 10 μm and 50 mm; and/or the distance between the large constriction and the small constriction along the fluid flow direction is between 50 μm and 5 mm; and/or the minimum dimension of the cross-section of the microfluidic channel between the large and small constrictions is between 7 μm and 19 μm; and/or the area of the cross-section of the microfluidic channel between the large and small constrictions is between 80 μm² and 1100 μm²; and the ratio between the minimum dimension of the cross-section of the large constriction and the minimum dimension of the cross-section of the small constriction is between 1.2 and 7; and/or the ratio between the area of the cross-section of the large constriction and the area of the cross-section of the small constriction is between 1.2 and 7; and/or the ratio between the minimum dimension of the cross-section of the large constriction and the minimum dimension of the cross-section of the microfluidic channel between the large and small constrictions is between 1.1 and 3.5; and/or the ratio between the area of the cross-section of the small constriction and the area of the cross-section of the microfluidic channel between the large and small constrictions is between 0.1 and 0.9.

In another preferred embodiment of the first aspect of the invention, the minimum dimension of the cross-section of the large constriction and/or the minimum dimension of the cross-section of the small constriction is between 2 μm and 20 μm; and the area of the cross-section of the large constriction and/or the area of the cross-section of the small constriction is between 25 μm² and 1200 μm²; and/or the large constriction and/or the small constriction extend over a length along the fluid flow direction between 10 μm and 50 mm; and/or the distance between the large constriction and the small constriction along the fluid flow direction is between 50 μm and 5 mm; and/or the minimum dimension of the cross-section of the microfluidic channel between the large and small constrictions is between 7 μm and 19 μm; and/or the area of the cross-section of the microfluidic channel between the large and small constrictions is between 80 μm² and 1100 μm²; and the ratio between the minimum dimension of the cross-section of the large constriction and the minimum dimension of the cross-section of the small constriction is between 1.2 and 7; and/or the ratio between the area of the cross-section of the large constriction and the area of the cross-section of the small constriction is between 1.2 and 7; and/or the ratio between the minimum dimension of the cross-section of the large constriction and the minimum dimension of the cross-section of the microfluidic channel between the large and small constrictions is between 1.1 and 3.5; and/or the ratio between the area of the cross-section of the small constriction and the area of the cross-section of the microfluidic channel between the large and small constrictions is between 0.1 and 0.9.

In another preferred embodiment of the first aspect of the invention, the minimum dimension of the cross-section of the large constriction and/or the minimum dimension of the cross-section of the small constriction is between 2 μm and 20 μm; and/or the area of the cross-section of the large constriction and the area of the cross-section of the small constriction is between 25 μm² and 1200 μm²; and/or the large constriction and/or the small constriction extend over a length along the fluid flow direction between 10 μm and 50 mm; and/or the distance between the large constriction and the small constriction along the fluid flow direction is between 50 μm and 5 mm; and/or the minimum dimension of the cross-section of the microfluidic channel between the large and small constrictions is between 7 μm and 19 μm; and/or the area of the cross-section of the microfluidic channel between the large and small constrictions is between 80 μm² and 1100 μm²; and the ratio between the minimum dimension of the cross-section of the large constriction and the minimum dimension of the cross-section of the small constriction is between 1.2 and 7; and/or the ratio between the area of the cross-section of the large constriction and the area of the cross-section of the small constriction is between 1.2 and 7; and/or the ratio between the minimum dimension of the cross-section of the large constriction and the minimum dimension of the cross-section of the microfluidic channel between the large and small constrictions is between 1.1 and 3.5; and/or the ratio between the area of the cross-section of the small constriction and the area of the cross-section of the microfluidic channel between the large and small constrictions is between 0.1 and 0.9.

In yet another preferred embodiment of the first aspect of the invention, the minimum dimension of the cross-section of the large constriction and/or the minimum dimension of the cross-section of the small constriction is between 2 μm and 20 μm; and/or the area of the cross-section of the large constriction and/or the area of the cross-section of the small constriction is between 25 μm² and 1200 μm²; and the large constriction and/or the small constriction extend over a length along the fluid flow direction between 10 μm and 50 mm; and/or the distance between the large constriction and the small constriction along the fluid flow direction is between 50 μm and 5 mm; and/or the minimum dimension of the cross-section of the microfluidic channel between the large and small constrictions is between 7 μm and 19 μm; and/or the area of the cross-section of the microfluidic channel between the large and small constrictions is between 80 μm² and 1100 μm²; and the ratio between the minimum dimension of the cross-section of the large constriction and the minimum dimension of the cross-section of the small constriction is between 1.2 and 7; and/or the ratio between the area of the cross-section of the large constriction and the area of the cross-section of the small constriction is between 1.2 and 7; and/or the ratio between the minimum dimension of the cross-section of the large constriction and the minimum dimension of the cross-section of the microfluidic channel between the large and small constrictions is between 1.1 and 3.5; and/or the ratio between the area of the cross-section of the small constriction and the area of the cross-section of the microfluidic channel between the large and small constrictions is between 0.1 and 0.9.

In a further preferred embodiment of the first aspect of the invention, the minimum dimension of the cross-section of the large constriction and/or the minimum dimension of the cross-section of the small constriction is between 2 μm and 20 μm; and/or the area of the cross-section of the large constriction and/or the area of the cross-section of the small constriction is between 25 μm² and 1200 μm²; and/or the large constriction and the small constriction extend over a length along the fluid flow direction between 10 μm and 50 mm; and/or the distance between the large constriction and the small constriction along the fluid flow direction is between 50 μm and 5 mm; and/or the minimum dimension of the cross-section of the microfluidic channel between the large and small constrictions is between 7 μm and 19 μm; and/or the area of the cross-section of the microfluidic channel between the large and small constrictions is between 80 μm² and 1100 μm²; and the ratio between the minimum dimension of the cross-section of the large constriction and the minimum dimension of the cross-section of the small constriction is between 1.2 and 7; and/or the ratio between the area of the cross-section of the large constriction and the area of the cross-section of the small constriction is between 1.2 and 7; and/or the ratio between the minimum dimension of the cross-section of the large constriction and the minimum dimension of the cross-section of the microfluidic channel between the large and small constrictions is between 1.1 and 3.5; and/or the ratio between the area of the cross-section of the small constriction and the area of the cross-section of the microfluidic channel between the large and small constrictions is between 0.1 and 0.9.

In yet a further preferred embodiment of the first aspect of the invention, the minimum dimension of the cross-section of the large constriction and/or the minimum dimension of the cross-section of the small constriction is between 2 μm and 20 μm; and/or the area of the cross-section of the large constriction and/or the area of the cross-section of the small constriction is between 25 μm² and 1200 μm²; and/or the large constriction and/or the small constriction extend over a length along the fluid flow direction between 10 μm and 50 mm; and the distance between the large constriction and the small constriction along the fluid flow direction is between 50 μm and 5 mm; and/or the minimum dimension of the cross-section of the microfluidic channel between the large and small constrictions is between 7 μm and 19 μm; and/or the area of the cross-section of the microfluidic channel between the large and small constrictions is between 80 μm² and 1100 μm²; and the ratio between the minimum dimension of the cross-section of the large constriction and the minimum dimension of the cross-section of the small constriction is between 1.2 and 7; and/or the ratio between the area of the cross-section of the large constriction and the area of the cross-section of the small constriction is between 1.2 and 7; and/or the ratio between the minimum dimension of the cross-section of the large constriction and the minimum dimension of the cross-section of the microfluidic channel between the large and small constrictions is between 1.1 and 3.5; and/or the ratio between the area of the cross-section of the small constriction and the area of the cross-section of the microfluidic channel between the large and small constrictions is between 0.1 and 0.9.

In another preferred embodiment of the first aspect of the invention, the minimum dimension of the cross-section of the large constriction and/or the minimum dimension of the cross-section of the small constriction is between 2 μm and 20 μm; and/or the area of the cross-section of the large constriction and/or the area of the cross-section of the small constriction is between 25 μm² and 1200 μm²; and/or the large constriction and/or the small constriction extend over a length along the fluid flow direction between 10 μm and 50 mm; and/or the distance between the large constriction and the small constriction along the fluid flow direction is between 50 μm and 5 mm; and the minimum dimension of the cross-section of the microfluidic channel between the large and small constrictions is between 7 μm and 19 μm; and/or the area of the cross-section of the microfluidic channel between the large and small constrictions is between 80 μm² and 1100 μm²; and the ratio between the minimum dimension of the cross-section of the large constriction and the minimum dimension of the cross-section of the small constriction is between 1.2 and 7; and/or the ratio between the area of the cross-section of the large constriction and the area of the cross-section of the small constriction is between 1.2 and 7; and/or the ratio between the minimum dimension of the cross-section of the large constriction and the minimum dimension of the cross-section of the microfluidic channel between the large and small constrictions is between 1.1 and 3.5; and/or the ratio between the area of the cross-section of the small constriction and the area of the cross-section of the microfluidic channel between the large and small constrictions is between 0.1 and 0.9.

In yet another preferred embodiment of the first aspect of the invention, the minimum dimension of the cross-section of the large constriction and/or the minimum dimension of the cross-section of the small constriction is between 2 μm and 20 μm; and/or the area of the cross-section of the large constriction and/or the area of the cross-section of the small constriction is between 25 μm² and 1200 μm²; and/or the large constriction and/or the small constriction extend over a length along the fluid flow direction between 10 μm and 50 mm; and/or the distance between the large constriction and the small constriction along the fluid flow direction is between 50 μm and 5 mm; and/or the minimum dimension of the cross-section of the microfluidic channel between the large and small constrictions is between 7 μm and 19 μm; and the area of the cross-section of the microfluidic channel between the large and small constrictions is between 80 μm² and 1100 μm²; and the ratio between the minimum dimension of the cross-section of the large constriction and the minimum dimension of the cross-section of the small constriction is between 1.2 and 7; and/or the ratio between the area of the cross-section of the large constriction and the area of the cross-section of the small constriction is between 1.2 and 7; and/or the ratio between the minimum dimension of the cross-section of the large constriction and the minimum dimension of the cross-section of the microfluidic channel between the large and small constrictions is between 1.1 and 3.5; and/or the ratio between the area of the cross-section of the small constriction and the area of the cross-section of the microfluidic channel between the large and small constrictions is between 0.1 and 0.9.

In a further preferred embodiment of the first aspect of the invention, the minimum dimension of the cross-section of the large constriction and/or the minimum dimension of the cross-section of the small constriction is between 2 μm and 20 μm; and/or the area of the cross-section of the large constriction and/or the area of the cross-section of the small constriction is between 25 μm² and 1200 μm²; and/or the large constriction and/or the small constriction extend over a length along the fluid flow direction between 10 μm and 50 mm; and/or the distance between the large constriction and the small constriction along the fluid flow direction is between 50 μm and 5 mm; and/or the minimum dimension of the cross-section of the microfluidic channel between the large and small constrictions is between 7 μm and 19 μm; and/or the area of the cross-section of the microfluidic channel between the large and small constrictions is between 80 μm² and 1100 μm²; and the ratio between the minimum dimension of the cross-section of the large constriction and the minimum dimension of the cross-section of the small constriction is between 1.2 and 7; and the ratio between the area of the cross-section of the large constriction and the area of the cross-section of the small constriction is between 1.2 and 7; and/or the ratio between the minimum dimension of the cross-section of the large constriction and the minimum dimension of the cross-section of the microfluidic channel between the large and small constrictions is between 1.1 and 3.5; and/or the ratio between the area of the cross-section of the small constriction and the area of the cross-section of the microfluidic channel between the large and small constrictions is between 0.1 and 0.9.

In another preferred embodiment of the first aspect of the invention, the minimum dimension of the cross-section of the large constriction and/or the minimum dimension of the cross-section of the small constriction is between 2 μm and 20 μm; and/or the area of the cross-section of the large constriction and/or the area of the cross-section of the small constriction is between 25 μm² and 1200 μm²; and/or the large constriction and/or the small constriction extend over a length along the fluid flow direction between 10 μm and 50 mm; and/or the distance between the large constriction and the small constriction along the fluid flow direction is between 50 μm and 5 mm; and/or the minimum dimension of the cross-section of the microfluidic channel between the large and small constrictions is between 7 μm and 19 μm; and/or the area of the cross-section of the microfluidic channel between the large and small constrictions is between 80 μm² and 1100 μm²; and the ratio between the minimum dimension of the cross-section of the large constriction and the minimum dimension of the cross-section of the small constriction is between 1.2 and 7; and/or the ratio between the area of the cross-section of the large constriction and the area of the cross-section of the small constriction is between 1.2 and 7; and the ratio between the minimum dimension of the cross-section of the large constriction and the minimum dimension of the cross-section of the microfluidic channel between the large and small constrictions is between 1.1 and 3.5; and/or the ratio between the area of the cross-section of the small constriction and the area of the cross-section of the microfluidic channel between the large and small constrictions is between 0.1 and 0.9.

In a further preferred embodiment of the first aspect of the invention, the minimum dimension of the cross-section of the large constriction and/or the minimum dimension of the cross-section of the small constriction is between 2 μm and 20 μm; and/or the area of the cross-section of the large constriction and/or the area of the cross-section of the small constriction is between 25 μm² and 1200 μm²; and/or the large constriction and/or the small constriction extend over a length along the fluid flow direction between 10 μm and 50 mm; and/or the distance between the large constriction and the small constriction along the fluid flow direction is between 50 μm and 5 mm; and/or the minimum dimension of the cross-section of the microfluidic channel between the large and small constrictions is between 7 μm and 19 μm; and/or the area of the cross-section of the microfluidic channel between the large and small constrictions is between 80 μm² and 1100 μm²; and the ratio between the minimum dimension of the cross-section of the large constriction and the minimum dimension of the cross-section of the small constriction is between 1.2 and 7; and/or the ratio between the area of the cross-section of the large constriction and the area of the cross-section of the small constriction is between 1.2 and 7; and/or the ratio between the minimum dimension of the cross-section of the large constriction and the minimum dimension of the cross-section of the microfluidic channel between the large and small constrictions is between 1.1 and 3.5; and the ratio between the area of the cross-section of the small constriction and the area of the cross-section of the microfluidic channel between the large and small constrictions is between 0.1 and 0.9.

In yet a further preferred embodiment of the first aspect of the invention, the minimum dimension of the cross-section of the large constriction and the minimum dimension of the cross-section of the small constriction is between 2 μm and 20 μm; and/or the area of the cross-section of the large constriction and the area of the cross-section of the small constriction is between 25 μm² and 1200 μm²; and/or the large constriction and the small constriction extend over a length along the fluid flow direction between 10 μm and 50 mm; and/or the distance between the large constriction and the small constriction along the fluid flow direction is between 50 μm and 5 mm; and/or the minimum dimension of the cross-section of the microfluidic channel between the large and small constrictions is between 7 μm and 19 μm; and/or the area of the cross-section of the microfluidic channel between the large and small constrictions is between 80 μm² and 1100 μm²; and the ratio between the minimum dimension of the cross-section of the large constriction and the minimum dimension of the cross-section of the small constriction is between 1.2 and 7; and/or the ratio between the area of the cross-section of the large constriction and the area of the cross-section of the small constriction is between 1.2 and 7; and/or the ratio between the minimum dimension of the cross-section of the large constriction and the minimum dimension of the cross-section of the microfluidic channel between the large and small constrictions is between 1.1 and 3.5; and/or the ratio between the area of the cross-section of the small constriction and the area of the cross-section of the microfluidic channel between the large and small constrictions is between 0.1 and 0.9.

In another preferred embodiment of the first aspect of the invention, the minimum dimension of the cross-section of the large constriction and the minimum dimension of the cross-section of the small constriction is between 2 μm and 20 μm; and the area of the cross-section of the large constriction and the area of the cross-section of the small constriction is between 25 μm² and 1200 μm²; and the large constriction and the small constriction extend over a length along the fluid flow direction between 10 μm and 50 mm; and the distance between the large constriction and the small constriction along the fluid flow direction is between 50 μm and 5 mm; and the minimum dimension of the cross-section of the microfluidic channel between the large and small constrictions is between 7 μm and 19 μm; and the area of the cross-section of the microfluidic channel between the large and small constrictions is between 80 μm² and 1100 μm²; and the ratio between the minimum dimension of the cross-section of the large constriction and the minimum dimension of the cross-section of the small constriction is between 1.2 and 7; and the ratio between the area of the cross-section of the large constriction and the area of the cross-section of the small constriction is between 1.2 and 7; and the ratio between the minimum dimension of the cross-section of the large constriction and the minimum dimension of the cross-section of the microfluidic channel between the large and small constrictions is between 1.1 and 3.5; and the ratio between the area of the cross-section of the small constriction and the area of the cross-section of the microfluidic channel between the large and small constrictions is between 0.1 and 0.9.

FIG. 3 shows a schematic diagram of a preferred embodiment in accordance with the first aspect of the invention. The microfluidic channel (100) comprises an inlet (101) and an outlet (102), as defined by the flow direction (103) of the suspension solution (108) comprising a cell/cells (106) and cargo molecules (107). The microfluidic channel comprises a first constriction (104), which is larger than the average cell diameter (110), and which is adapted to apply hydrodynamic forces to the cell as it passes through, keeping low Reynolds numbers (about 7-20). Reynolds number (Re), is defined as the ratio of inertial forces to viscous forces, it is calculated as Re=4Q/Pv, where Q, is the volumetric flow rate (m³ s⁻¹) in the first constriction, P is the perimeter of the first constriction (m) and v is the kinematic viscosity of the surrounding medium (m² s⁻¹). The first deformation is done to achieve initial membrane permeabilization (109). The second constriction (105) on the other hand is equal to or smaller than the average cell diameter and adapted to apply contact-based compression forces to the cell. The second compression is done to increase the number and size of membrane pores.

In accordance with another preferred embodiment of the first aspect of the invention said at least one microfluidic channel comprises three constrictions and the third of said three constrictions has a cross-section that is smaller than the average cross-section of said cell and adapted to apply contact-based compression forces to said cell while allowing said cell to pass through said constriction and wherein the cross-section of the third constriction is smaller than the cross-section of the second constriction.

FIG. 4 shows a schematic diagram of a preferred embodiment in accordance with the first aspect of the invention. The microfluidic channel (100) comprises an inlet (101) and an outlet (102), as defined by the flow direction (103) of the suspension solution (108) comprising a cell/cells (106) and cargo molecules (107). The microfluidic channel comprises a first constriction (111), which is larger than the average cell diameter (110), and which is adapted to apply hydrodynamic forces to the cell as it passes through. The second constriction (112) and the third constriction (113) are equal to or smaller than the average cell diameter and adapted to apply contact-based compression forces to the cell. Here, contact-based compression is done gradually to increase the pore number and size without compromising cell viability.

In accordance with a yet further preferred embodiment of the first aspect of the invention the constrictions of said microfluidic device are arranged with descending-sized cross-sections with regard to the flow direction of the cells or the constrictions of said microfluidic device are arranged with ascending-sized cross-sections with regard to the flow direction of the cells. Both arrangements provide a gradual and gentle cell deformation.

FIG. 5 shows a schematic diagram of another preferred embodiment in accordance with the first aspect of the invention. The microfluidic channel (100) comprises an inlet (101) and an outlet (102), as defined by the flow direction (103) of the suspension solution (108) comprising a cell/cells (106) and cargo molecules (107). The microfluidic channel comprises a first constriction (114) and a second constriction (115), which are equal to or smaller than the average cell diameter (110), and which are adapted to apply contact-based compression forces to the cell as it passes through. The third constriction (116) is larger than smaller than the average cell diameter and adapted to apply hydrodynamic forces to the cell. The first of said constrictions applies the strongest deforming force to the cell. Due to the narrow first constriction, the cell membrane is strongly permeabilized. The second and third constriction will delay the membrane recovery time and allow diffusion of big size cargo into the cytoplasm.

In accordance with further preferred embodiment of the first aspect of the invention, the microfluidic device of the invention comprises at least 10 microfluidic channels, which are arranged in parallel to provide for parallel connection.

The use of such parallel configuration does not only increase the overall throughput, but also reduces the consequence of blockages of cells developing in one or more of the microfluidic channels.

In alteration of the first aspect of the invention, progressive mechanoporation is achieved without channels, e.g. by rotating parallel plate geometry. In such cases, the shear rate can be controlled by adjusting the rotation frequency of the rotating plate and contact-based compression forces can be controlled by adjusting the distance between the plates. A one directional movement of the plates for decreasing the distance will result in contact-based compression, while oscillatory movement of the plates upwards and downwards, applies compression by contact and/or by hydrodynamic forces.

In accordance with the embodiment as described above, a procedure for intracellular delivery may be defined to alternate between the different modes of applying hydrodynamic shear forces, contact-based forces and/or hydrodynamic pressure forces.

It is important to provide a microfluidic platform to allow the operation under a broad range of pressure/flow conditions in a reliable, reproducible and easy to use way.

Hence, in accordance with another preferred embodiment of the first aspect of the invention, the flow inside the microfluidic device is controlled by either a pressure-driven (constant pressure) or a syringe-driven (constant flow) system.

As syringe-driven systems are generally limited by longer response and stabilization time and high fluctuations, pressure-driven flow provides faster response and stabilization. Thus, pressure-driven systems are preferably used to process small sample volumes in a short time, where a control of the flow rate is needed to ensure process stability over the experimental time.

In accordance with a yet further preferred embodiment of the first aspect of the invention, the microfluidic device is made of polydimethylsiloxane (PDMS). However, any other material suitable for etching micron scaled features is suitable, including, but not limited to silicon, metal, plastic (e.g. thermoplastic, fluoroplastic) and glass. For real time analysis of deformation, preferably transparent material (e.g. PDMS, glass, thermoplastic and the like) is used.

In accordance with still a further preferred embodiment of the first aspect of the invention, the microfluidic device of the invention is integrated into a chip.

In a second aspect the invention relates to a system comprising the microfluidic device of the invention and further comprising a fluid pressure regulator and/or a flow sensor which is connected to the inlet of the microfluidic device.

The definitions and preferred embodiments of the first aspect of the invention apply mutatis mutandis to the second aspect of the invention.

The “fluid pressure regulator” allows the control of pressure-driven flow through the microfluidic device, the “flow sensor” allows monitoring the flow of fluid in a channel.

In a preferred embodiment of the second aspect of the invention, the microfluidic device comprises an inlet chamber, which is in fluid communication with the inlet of the microfluidic device.

In accordance with the preferred embodiment described above, said fluid pressure regulator is connected to the inlet chamber and/or said flow sensor connects the inlet chamber and the microfluidic device inlet.

The term “chamber” as used herein generally relates to a three-dimensional hollow structure which has a top and bottom and is surrounded by walls, enclosing a volume suitable to store and/or convey a fluid i.e. the cell suspension solution and cargo molecules. The term “inlet chamber” refers to a chamber which is in fluid communication with the inlet of the microfluidic device.

In another preferred embodiment of the second aspect of the invention, the microfluidic device comprises an outlet chamber, which is in fluid communication with the outlet of the microfluidic device.

In accordance with the above-mentioned embodiment, the mechanoporated cells may be collected in said outlet chamber.

In an even more preferred embodiment of the second aspect of the invention, the fluid pressure regulator is adapted to provide a fluid flow at a velocity higher than 0.1 m/s and more preferably between 0.5 and 1.5 m/s through the at least two cell-deforming constrictions of the microfluidic channel.

In a particularly preferred embodiment, the fluid pressure regulator is a pressure-based microfluidic controller (see e.g. Example 1).

In accordance with another preferred embodiment of the second aspect of the invention, a filter is positioned in the direction of the flow after the microfluidic device inlet. Such a filter helps to avoid blockages of debris in the channels. The term “debris” as used herein can be cell debris, extracellular matrix, tissue components and contaminating environmental entities (e.g. micrometer-sized particles and fibers and their agglomerate) with size bigger than the typical channel size.

At variance with the above-mentioned preferred embodiment, said filter may be integrated into the inlet chamber.

Furthermore, in accordance with the above-mentioned preferred embodiment, the filter may be compromised of a periodic pillar structure or may be a porous membrane.

In a particularly preferred embodiment as shown in FIG. 7, the system consists of a fluid pressure regulator, a flow sensor and a microfluidic device, interconnected by at least two chambers filled respectively with a cell culture medium and a cell suspension solution.

A system in accordance with the second aspect of the invention comprising a pressure-based microfluidic controller and a flow sensor is described in the examples herein below and is particularly advantageous, as both the operating pressure and flow rate can be controlled and ensure reliable and reproducible cell treatment.

Thus, in accordance with a further embodiment of the second aspect of the invention, the fluid pressure regulator regulates the fluid flow through the microfluidic channel, thereby applying a shear force on the cell for >1 ms at <1500 Pa, preferably for a minimum of 10 ms at 100-500 Pa, when passing the suspension solution through the large cell-deforming constriction.

In a third aspect, the present invention relates to a method for delivery of cargo molecules into a cell, the method comprising: a) passing a suspension solution comprising said cell and said cargo molecules through the microfluidic device of the invention whereby said cargo molecules are delivered into said cell; and b) collecting cells into which said cargo molecules have been delivered.

It is understood that the definitions and embodiments as described above in the context of the first and the second aspect of the invention also apply, in as far as possible, mutatis mutandis to the third aspect of the present invention and vice versa.

The method of the third aspect of the invention is directed to delivering cargo molecules into a cell by the device of the invention. Accordingly, the present invention also relates to the use of the device of the invention for delivering cargo molecules into a cell.

The step of passing a suspension solution comprising said cell and said cargo molecules through the microfluidic device of the invention is achieved by, for example, a pressure pump, a gas cylinder, a compressor, a vacuum pump, a syringe, a syringe pump, a peristaltic pump, a manual syringe, a pipette, a piston, a capillary actor or gravity.

By passing the suspension solution comprising said cell and said cargo molecules through the microfluidic device of the invention, pores are introduced into the cell membrane and/or the diameter of pores in the cell membrane is enhanced by cell deformation caused by hydrodynamic and contact-based compression forces which are applied to said cell by the constrictions of the microfluidic device of the invention. These pores allow and/or facilitate the delivery of cargo molecules from outside the cell to move into the cell.

In a preferred embodiment of the third aspect of the invention, said cell has a Young's modulus of ≤3.5 kPa.

As can be taken from the examples herein below, the use of progressive cell membrane mechanoporation for intracellular delivery as described herein resulted high delivery efficiency and high cell viability, specifically into cells with a Young's modulus of ≤3.5 kPa. However, progressive cell membrane mechanoporation as described herein may also be used to deliver cargo molecules into cells with a Young's modulus above 3.5 kPa with high delivery efficiency and high cell viability by adapting the cell velocity, the viscosity of the medium and/or the channel geometry, including the arrangement and dimensions of the constrictions.

In accordance with a further preferred embodiment of the third aspect of the invention, the method further comprises regulating the fluid flow through the microfluidic channel and/or measuring the flow rate through the microfluidic channel. As discussed herein above, such regulation can be achieved by using a fluid pressure regulator. By regulating the fluid flow, the duration of the deforming force applied to the cell can be varied. Changing the fluid flow changes the cell velocity and consequently the time that the cell is present in the microfluidic channel.

In accordance with another preferred embodiment of the third aspect of the invention, the method comprises passing the suspension solution at a velocity higher than 0.1 m/s and more preferably between 0.5 and 1.5 m/s through the at least two cell-deforming constrictions of the microfluidic channel.

In accordance with another embodiment of the third aspect of the invention, passing the suspension solution through the large cell-deforming constriction applies a shear force on the cell for >1ms at <1500Pa, preferably for a minimum of 10 ms at 100-500 Pa.

In yet a further preferred embodiment of the third aspect of the invention, the method comprises passing the suspension solution through the large cell-deforming constriction which applies a shear force on the cell for a minimum of 10 ms at 100-500 Pa; and/or passing the suspension solution through the small cell-deforming constriction which applies a compression force and optionally a shear force on the cell for a maximum of 1 ms, wherein the shear force is 1500-6500 Pa.

In this context it is important to note that in previous methods, as e.g. described in US 2014/287509, the cells are subjected to a pulse of shear stress (approximately 100 μs) in cases where the constriction is larger than the diameter of the cell. In contrast and in accordance with the embodiments described above, during progressive mechanoporation, the cells are subjected to prolonged shear stress during the step of pre-deformation (i.e. when passing through the large cell deforming constriction). Such prolonged pre-deformation is thought to avoid mechanical shock on cells that approach narrower constrictions and consequently allows deformation of cells in narrower constrictions, wherein higher shear forces ranging between 2500-6000 Pa can be applied to the cells without impacting cell viability.

Hence, by varying the operating pressure, cell velocity and thus also the duration of the deforming force applied to the cell when it passes through the at least two constrictions, i.e. degree of cell deformation, can be customized, allowing strong cell deformation (ratio between major and minor axis of the cell of 1.25 to 3.3) and subsequently high delivery efficiency while maintaining high cell viability. Real-time image-based detection may be used evaluate the degree of deformation in the first constriction and the flow rate may be adjusted accordingly to provide a certain value of deformation.

Hence, in accordance with a further embodiment of the third aspect of the present invention, the ratio between the major and the minor axis of the cell is between 1.25 and 3.3 as it passes through the large cell-deforming constriction; and/or the ratio between the major and the minor axis of the cell is between 4 and 12.5 as it passes through the small cell-deforming constriction.

In accordance with a yet further preferred embodiment of the third aspect of the invention, the cell is incubated for a sufficient time to allow recovery of the cell membrane.

The time sufficient to allow recovery of the cell membrane is dependent on the cell Young's Modulus and the supplements in the cell suspension solution. The time sufficient to allow recovery of the cell membrane can be altered by changing e.g. the medium composition and/or the temperature. For example, if Calcium is removed from the cell suspension solution, the process of cell membrane recovery will be slower. Preferably, the time sufficient to allow recovery of the cell membrane ranges between 10 s to 10 min.

In another particularly preferred embodiment of the third aspect of the invention, the cell is a eukaryotic cell.

In accordance with another preferred embodiment of the third aspect of the invention, the cargo molecules include small molecules, amino acids, proteins, nucleic acids, impermeable dyes, nano-carrier systems, CRISPR/Cas9 gene-editing complexes and combinations thereof.

The invention is herein described, by way of example only, with reference to the accompanying drawings for purposes of illustrative discussion of the preferred embodiments of the present invention.

Unless otherwise defined, all technical and scientific terms used herein have the same meaning as commonly understood by one of ordinary skill in the art to which this invention belongs. In case of conflict, the patent specification including definitions, will prevail.

Regarding the embodiments characterized in this specification, in particular in the claims, it is intended that each embodiment mentioned in a dependent claim is combined with each embodiment of each claim (independent or dependent) said dependent claim depends from. For example, in case of an independent claim 1 reciting 3 alternatives A, B and C, a dependent claim 2 reciting 3 alternatives D, E and F and a claim 3 depending from claims 1 and 2 and reciting 3 alternatives G, H and I, it is to be understood that the specification unambiguously discloses embodiments corresponding to combinations A, D, G; A, D, H; A, D, I; A, E, G; A, E, H; A, E, I; A, F, G; A, F, H; A, F, I; B, D, G; B, D, H; B, D, I; B, E, G; B, E, H; B, E, I; B, F, G; B, F, H; B, F, I; C, D, G; C, D, H; C, D, I; C, E, G; C, E, H; C, E, I; C, F, G; C, F, H; C, F, I, unless specifically mentioned otherwise.

Similarly, and also in those cases where independent and/or dependent claims do not recite alternatives, it is understood that if dependent claims refer back to a plurality of preceding claims, any combination of subject-matter covered thereby is considered to be explicitly disclosed. For example, in case of an independent claim 1, a dependent claim 2 referring back to claim 1, and a dependent claim 3 referring back to both claims 2 and 1, it follows that the combination of the subject-matter of claims 3 and 1 is clearly and unambiguously disclosed as is the combination of the subject-matter of claims 3, 2 and 1. In case a further dependent claim 4 is present which refers to any one of claims 1 to 3, it follows that the combination of the subject-matter of claims 4 and 1, of claims 4, 2 and 1, of claims 4, 3 and 1, as well as of claims 4, 3, 2 and 1 is clearly and unambiguously disclosed.

The above considerations apply mutatis mutandis to all appended claims.

### BRIEF DESCRIPTION OF THE DRAWINGS

FIG. 1 shows a schematic microfluidic channel where cells are deformed due to high shear exerted by the fluid. Figure taken from Kizer et al., 2019 (¹⁸).

FIG. 2 shows a schematic microfluidic channel where cells are deformed due to contact-based compression exerted by the walls of the channel. Figure taken from Liu et al., 2018(¹⁷).

FIG. 3 shows an exemplary configuration of a microfluidic channel consisting of first constriction wherein cells are deformed due to hydrodynamic forces and a second constriction where cells are deformed due to contact-based compression forces when passing through the constrictions.

FIG. 4 shows an exemplary configuration of a microfluidic channel consisting of three constrictions, wherein the first constriction applies hydrodynamic forces to the passing cells and the second and third constrictions apply contact-based compression forces to the passing cells.

FIG. 5 shows an exemplary configuration of a microfluidic channel consisting of three constrictions, wherein the first and second constrictions apply contact-based compression forces to the passing cells and wherein the third constriction applies hydrodynamic forces to the passing cells.

FIG. 6 shows a schematic representation of the progressive cell membrane mechanoporation during cell flow inside a single microfluidic channel and cell membrane recovery in a collection tube (reservoir) according to the first aspect of the invention. The microfluidic channel comprises three deformation regions: Low (LDR), Medium (MDR) and High Deformation Region (HDR). For each region, length (L) and width (W) of the corresponding channel are indicated. The arrows in the MDR, HDR and before the outlet, indicate the cell volume exchange resulting in convective molecular transport into cell cytoplasm. The arrows in the reservoir show the diffusion of molecules from the surrounding medium to the cell cytosol before cell membrane repair.

FIG. 7 shows a schematic representation of a microfluidic system for progressive mechanoporation, wherein the set-up consists of a fluid pressure regulator which is operated by a software and connected to a chamber containing cell culture media (CMV) which is further connected to a flow sensor via a further chamber filled with cell culture media (CMT). Another chamber filled with cell suspension solution (CST) is attached between the flow sensor and microfluidic device inlet. To collect the mechanoporated cells, a chamber is in fluid communication with the outlet of the device.

FIG. 8: a) Pressure stabilization over the period of time for four different operating pressures (3, 4 and 5 bar). The gradient grey zone shows the device delamination for operating pressure higher than 5.5 bar. b) Flow rate at the device inlet, Qᵢ, as function of the applied pressure for four different device geometries (L_{c}_W_{c}=40_6; 60_6; 40_4; 60_4).

FIG. 9: Analysis of cell size and elasticity. a) Cell diameter distribution for three different cell types: HeLa K, RPE-1 and U2OS. The mean and SDs are plotted. b) Young's modulus distribution of Hela K, RPE-1 and U2OS cells measured by real-time deformability cytometry (RT-DC).

FIG. 10: Intracellular delivery of small cargo into cells comparable in size and different elasticity. a) Delivery efficiency of 4 kDa FITC-dextran in HeLa K and RPE-1 cells, obtained by using 40 μm-6 μm (L_{c}-W_{c}) (top) and 60 μm-6 μm (L_{c}-W_{c}) (bottom) devices. The delivery efficiency was measured by FACS for the following conditions: not treated cells (Ctrl); cells treated with operating pressure of 3 bar and 5 bar. The symbol (+) represents the addition of 4 kDa FITC-dextran to the cell suspension. Individual measurements (circles) and mean values (line) are reported. Unpaired T-test was performed to test for significance of differences between 3 bar and 5 bar, p values were calculated and a p value higher than 0.05 was considered non-significant. b) Cell viability of HeLa K and RPE-1 cells represented as Propidium Iodide (PI) negative cells (top), measured by FACS analysis on cells stained with PI right after PM in 40 μm-6 μm and 60 μm-6 μm (L_{c}-W_{c}) devices (pressure 5 bar) and without PM (Ctrl). Fold increase in cell number (bottom) comparing HeLa K and RPE-1 cells 24 hours after PM in 40 μm-6 μm and 60 μm-6 μm (L_{c}-W_{c}) devices and without PM (Ctrl) measured by bright-field microscopy. The mean values and SD of three independent experiments are plotted. Kruskal-Wallis one-way analysis of variance was performed to test for significance of differences, p values were calculated. A p value higher than 0.05 was considered non-significant.

FIG. 11: Progressive mechanoporation in narrower constriction enhances delivery efficiency of small cargo into stiffer cells. a) Delivery efficiency of 4 kDa FITC-dextran in HeLa K and RPE-1 cells, obtained by using 40 μm-4 μm (L_{c}-W_{c}) (top) and 60 μm-4 μm (L_{c}-W_{c}) (bottom) devices. The delivery efficiency was measured by FACS for the following conditions: not treated cells (Ctrl); cells treated with operating pressure of 3 bar and 5 bar. The symbol (+) represents the addition of 4 kDa FITC-dextran to the cell suspension. Individual measurements (circles) and mean values (line) are reported. Unpaired T-test was performed to test for significance of differences between 3 bar and 5 bar, p values were calculated and a p value higher than 0.05 was considered non-significant. b) Cell viability of HeLa K and RPE-1 cells represented as Propidium Iodide (PI) negative cells (top), measured by FACS analysis on cells stained with PI right after PM in 40 μm-4 μm and 60 μm-4 μm (L_{c}-W_{c}) devices (pressure 3 bar) and without PM (Ctrl). Fold increase in cell number (bottom) comparing HeLa K and RPE-1 cells 24 hours after PM in 40 μm-4 μm and 60 μm-4 μm (L_{c}-W_{c}) devices and without PM (Ctrl) measured by bright-field microscopy. The mean values and SD of three independent experiments are plotted and p values are indicated. Kruskal-Wallis one-way analysis of variance was performed to test for significance of differences, a p value higher than 0.05 was considered non-significant.

FIG. 12: Intracellular delivery of bigger molecules in size range of biologically relevant cargo. Delivery efficiency of 70 kDa FITC-dextran in HeLa K (top) and RPE-1 (bottom) cells, obtained by using 40 μm-4 μm and 60 μm-4 μm (L_{c}-W_{c}) devices. The delivery efficiency was measured by FACS for the following conditions: not treated cells (Ctrl); cells treated with operating pressure of 3 bar. The symbol (+) represents the addition of 70 kDa FITC-dextran to the cell suspension. Individual measurements (circles) and mean values (line) are reported.

FIG. 13: Successful delivery of Cas9-sgRNA RNPs. a) Scheme of the Cas9-NLS protein-sgRNA RNPs delivery into U2OS cells (D0=Day 0), cell splitting (D1=Day 1) and the microscopy (D3=Day 3) and FACS (D4=Day 4) analysis. b) Delivery efficiency of Cas9-NLS protein-sgRNA RNPs targeting GFP (GFP targeting) or non-targeting in U2OS, obtained by using 40 μm-4 μm and 60 μm-4 μm (L_{c}-W_{c}) devices with operating pressure of 3 bar and analysed by FACS as percentage of GFP negative cells. Individual measurements (circles) and mean values (line) are reported. The data were obtained from two independent experiments (with three technical repeats for Cas9-sgRNA RNPs targeting GFP and two technical repeats for Cas9-sgRNA RNPs non-targeting within one independent experiment). c) Fluorescent images of cells after delivery of Non-targeting or GFP-targeting Cas9-sgRNA RNPs as indicated in (a), by using 40 μm-4 μm (L_{c}-W_{c}) (top) and 60μm-4μm (L_{c}-W_{c}) (bottom) devices. DAPI was used for DNA staining. The scale bar is 20 μm.

FIG. 14: Bright field image of a microfluidic device, 40 μm-6 μm (L_{c}-W_{c}), showing HeLa K cells flowing through 9 microfluidic channels under an applied pressure of 5 bar. Scale bar: 50 μm.

FIG. 15: (a) RT-DC measurements of individual cell types (HeLa K, RPE-1, U2OS and BJs) representing the deformation for each cell event with its corresponding measured cell area (μm²). Total number of measured cell events is indicated. (b) Comparison of contour plot of cell deformation versus area for the four cell types (HeLa K, RPE-1, U2OS and BJs).

FIG. 16: Examples of fluorescence intensity histograms obtained by FACS analysis of HeLa K (a) and RPE-1 (b) cells exposed to 4 kDa FITC-dextran without PM (Ctrl+FITC-dextran) or with PM using 40 μm-6 μm (L_{c}-W_{c}) and 40 μm-4 μm (L_{c}-W_{c}) devices under an applied pressure of 3 bar. In individual measurements, up to 5,000 cells were analysed. Cells with or without PM were exposed to the same concentration of 4 kDa FITC-dextran over the same period of time. To distinguish FITC positive cells (FITC+) due to delivery via PM, we excluded cell counts resulting from autofluorescence, endocytosis and surface binding (Ctrl+FITC-dextran). The gating strategy for FITC+cells was defined that less than 1% of Ctrl+FITC-dextran cells were classified as FITC+.

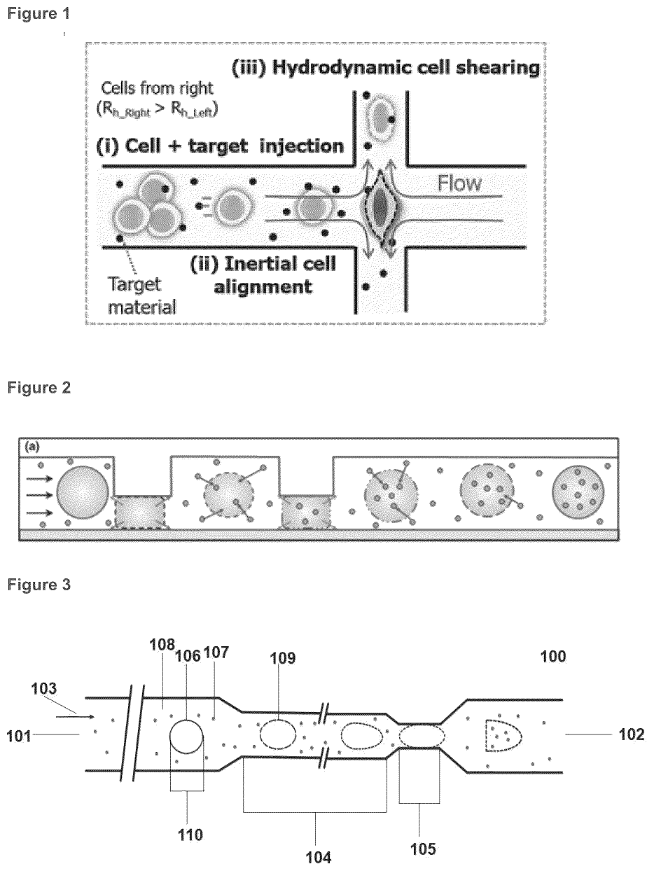

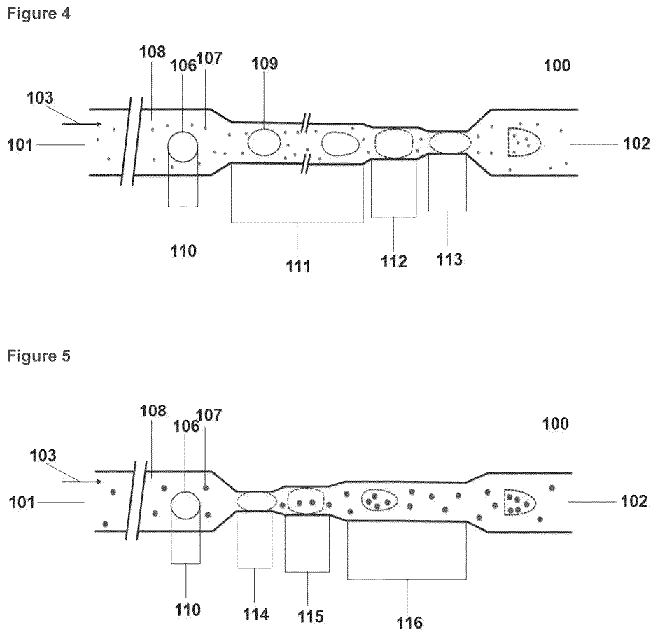

The examples illustrate the invention:

### Example 1: Materials and Methods

### Master Fabrication

The microfluidic device was designed using KLayout software. To avoid blockages in the channels and PDMS collapse, a filter was included as an array of circular pillars (diameter=100 μm) with periodicity of 50-200 μm after the inlet chamber. The master used for the fabrication of PDMS devices was realized through photolithography process (EVG®620 Automated NIL System) using AZ15nXT 450 cps photoresist (MicroChemicals GmbH). A 4″ silicon wafer was coated with the photoresist by spin coating at 850 rpm for 30 s (Laurell WS-650Hzb-23NPP-UD-3 spin coater). After coating, the wafer was exposed to UV light (550 mJ/cm2) through chromium photomask containing the channel geometry. After baking, the photoresist was developed using a solution of AZ 400K developer (MicroChemicals GmbH) (1:3 v/v, developer: distilled water) for 2 mins 30 s. The height of the fabricated structures was analysed using stylus profiler (Bruker DektakXT-A) and it was 18 μm. The prepared master template was functionalized with 1H,1H,2H,2H-Perfluorodecyltriethoxysilane (Sigma-Aldrich) in a desiccator for 12 h before using.

### Device Fabrication

The structures on the master were replicated on polydimethylsiloxane (PDMS) element by replica molding process. The base (Dow Corning Sylgard® 184) and curing agent were mixed in ratio 10:1, degassed, poured on the master and polymerized in the oven at 75° C. for 1.5 hrs. The cured PDMS was then peeled off and it was punched using biopsy puncher (Imtegra GmbH) in correspondence of the inlet (I.D. 1 mm) and the outlet (I.D. 1.5 mm) chambers. The punched PDMS replica was then bonded on glass coverslip (40×24 mm, thickness 2, Hecht) by activating their surface using air plasma process (50 W, 30 s, Gambetti, Tucano plasma reactor). The bonded PDMS device was incubated at 75° C. for 12 h.

### Microfluidic System

The microfluidic system included a pressure controller (Fluigent MFCS™-EX) equipped with a channel that is able to provide maximum pressure of 7 bar, operated by a software MAESFLO™. The pressure controller was connected via Tygon tubing (OD=4 mm, ID=2.5 mm) to a chamber containing cell culture media (CMV) which was further connected to a flow sensor (Fluigent Flow Rate Platform, XL) using FEP tubing (O.D.=1.5 mm, I.D.=500 μm, Kinesis GmbH) to fill the flow sensor with cell culture media. The device was filled with cell culture media from a tubing connected to the outlet to avoid any air bubbles in the system. Another FEP tubing (O.D.=1.5 mm, I.D.=500 μm, Kinesis GmbH) was filled with cell suspension (CST) whose one end was connected to the flow sensor (Flow unit—XL, Fluigent) and the other end was fitted inside the device inlet. The cell flow through the microfluidic device was activated and monitored by increasing the applied pressure through the microfluidic pressure controller while recording in real time the corresponding flow rate measured by the in-line flow sensor.

### Real-Time Deformability Cytometry (RT-DC)

The RT-DC measurements were performed by using the same setup and device described in a previous publication³². Briefly, the cells were trypsinised and were centrifuged at 100 g for 5 min. The supernatant was removed, and the cells were resuspended at a concentration of around 5×10⁶ cells/ml in 1×PBS buffer with 0.5% (w/v) methylcellulose having viscosity of 15 mPa·s. The cell samples were flowed through RT-DC devices with 30 μm square channel geometry at a flow rate of 0.16 μL/s. All the samples were processed at room temperature within 30 min of preparation. All analyses were done using the open-source software ShapeOut³⁶. The Young's moduli of the cells were calculated using ShapeOut after filtering out the events falling out of area range of 80 μm²-750 μm² and area ratio range of 0-1.05.

### Cell Culture

Cells were cultured according to standard mammalian tissue culture protocols and sterile techniques at 37° C. in 5% CO₂ and tested in regular intervals for mycoplasma. Site-specific integration of mRuby into one allele of endogenous PCNA and of mTurquoise2 into an allele of histone 3.1 of hTERT RPE-1 FRT/TO cells was achieved by rAAV-mediated homologous recombination as described previously²³ followed by selection of single positive integrands by flow cytometry.

HeLa K cells were cultured in DMEM (Gibco, Cat. no. 41966) supplemented with 10% (v/v) FBS (Gibco), 1% (v/v) Glutamax (Gibco), 1% (v/v) penicillin-streptomycin (Sigma-Aldrich) and 0.5 μg/ml amphotericin B (Sigma-Aldrich). hTERT RPE-1 FRT/TO cells were cultured in DMEM/F12 (Sigma-Aldrich, Cat. no. D6421) supplemented with 10% (v/v) FBS, 1% (v/v) Glutamax, 0.26% (v/v) sodium bicarbonate (Gibco), 1% (v/v) penicillin-streptomycin and 0.5 μg/ml Amphotericin B. U2OS cells were cultured in DMEM (Gibco, Cat. no. 31966047) supplemented with 10% (v/v) FBS and 1% (v/v) penicillin-streptomycin (Gibco).

Before and during PM, cells were kept in Leibovitz's L-15 media (Gibco, Cat. no. 21083027) supplemented with 10% (v/v) FBS, 1% (v/v) Glutamax, 1% (v/v) penicillin-streptomycin and 0.5 μg/ml amphotericin B (in case of U2OS without amphotericin B). Before the PM, cells were washed with PBS and detached with Trypsin-EDTA (0.05%) (Gibco). Trypsinisation was stopped by addition of appropriate cell culture media. After spinning down cells were re-suspended in L-15 media, filtered using the CellTrics filter with the 50 μm diameter (Sysmex) and counted. In case of experiments with HeLa K 20,000 cells per condition were used (except for determination of cell viability using propidium iodide staining where 50,000 cells were used), in case of hTERT RPE-1 50,000 cells and in case of U2OS 32,000 cells were used. After the PM, cells were spun down, washed and seeded in an appropriate fresh cell culture media or immediately analysed.

### Propidium Iodide Staining

For analysis of cell viability cells were taken right after the PM and cell suspension in L-15 media was supplemented with 1 μg/ml propidium iodide (Sigma-Aldrich) staining dead cells. The cell analyser BD LSRII (BD Biosciences) was used to detect propidium iodide (PI) positive/negative cells, up to 20,000 events were counted. Data were analysed in BD FACSDiva Software Version 8.0.2.

### Cell Proliferation Analysis

Cells after PM were plated in a 96 well plate (Greiner Bio-One), after 4 h cells were washed twice with PBS and imaging media (DMEM without phenol red and riboflavin—Gibco, cat.no. 041-96205M, supplemented with 10% (v/v) FBS, 1% (v/v) Glutamax, 1% (v/v) penicillin-streptomycin and 0.5 μg/ml amphotericin B) was added. Afterwards fluorescent microscopy imaging of histone 2B and histone 3.1 was performed using ImageXpress Micro XLS wide-field screening microscope (Molecular Devices) equipped with 10×, 0.5 numerical aperture Plan Apo air objective (Nikon). After 24 h cells were imaged again. Image analysis for counting number of nuclei based on histone 2B and histone 3.1 was done in MetaXpress software (Molecular Devices). The ratio between number of nuclei 24 h after PM and number of nuclei in time point 0 was calculated and it is called a fold increase in cell number.

### Fitc-Dextran Delivery

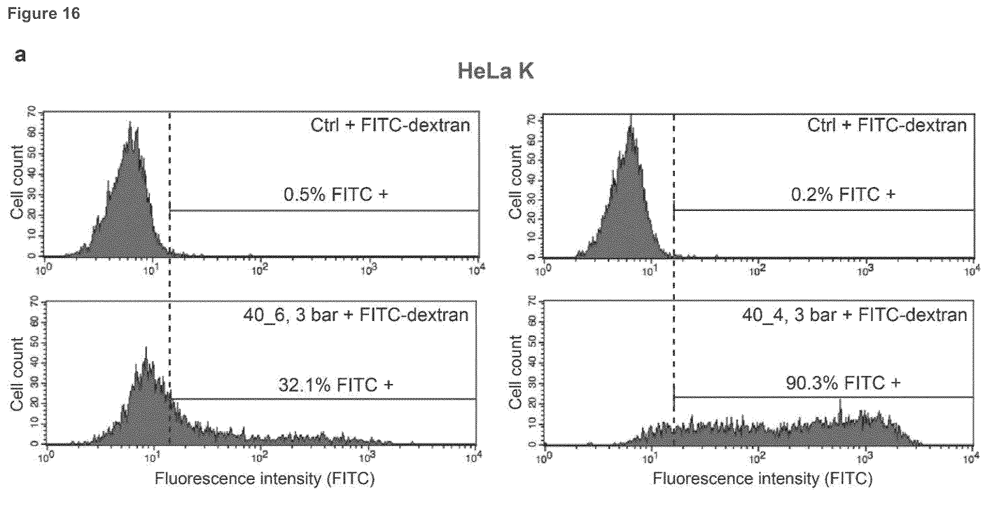

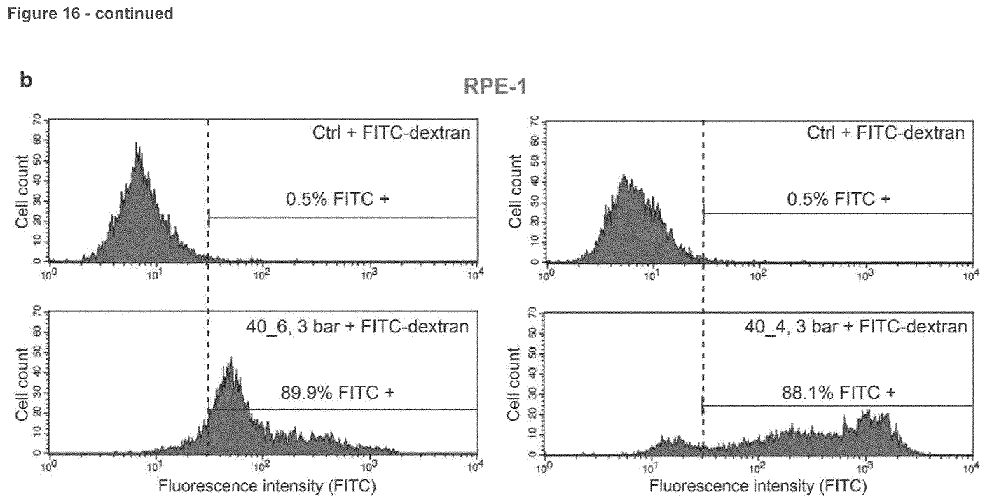

4 kDa FITC-dextran and 70 kDa FITC-dextran (Sigma-Aldrich) were used as model cargo. 4 kDa and 70 kDa FITC-dextran were dissolved in water and were used in the final concentration of 0.2 mg/ml. FITC-dextran was added to cell suspension right before flowing it through the device without any pre-incubation. To control delivery independent of PM, cell suspension was mixed with FITC-dextran and incubated for the same time as treated samples (Ctrl+FITC-dextran). The whole process of PM was done at room temperature within an hour. Cells after PM were plated in a 96 well plate and after 20 h cells were washed once with PBS, trypsinised and analysed. FITC-positive cells were detected using the cell analyser FACS Caliber™ (BD Biosciences), up to 5,000 total events were counted. To distinguish FITC positive cells due to delivery via PM, we excluded cell counts resulting from autofluorescence, endocytosis and surface binding (Ctrl+FITC-dextran) (FIG. 16). The gating strategy for FITC positive cells was defined that less than 1% of Ctrl+FITC-dextran cells were classified as FITC positive. The threshold for FITC positive cells was individually determined for each experiment respecting the gating strategy because contribution of delivery independent of PM as well as cell autofluorescence can vary between different experiments. Data were analysed in BD CellQuest™.

### Formation and Delivery of SpCas9NLS-gRNA Ribonucleoprotein (RNP)

SpCas9NLS protein (=wild-type Cas9 nuclease from Streptococcus pyogenes, fused with a C-terminal nuclear localization signal (NLS) (158.4 kDa), Eupheria Biotech 5,000 ng/μL) was diluted to 25 μM with HEPES/KCl buffer pH 7.25 (20 mM HEPES, 150 mM KCl, 1 mM DTT). sgRNA (custom made modified single guide RNA (32.4 kDa), Synthego corporation) was dissolved to 100 μM in Tris-EDTA-buffer pH 8.0 (Synthego corporation) and diluted to 25 μM with nuclease-free water (Synthego corporation).

sgRNA sequence ‘targeting GFP’:
(see SEQ ID NO: 1)
G*G*C*CACAAGUUCAGCGUGUC + Synthego modified
EZ Scaffold
sgRNA sequence ‘non-targeting’:
(see SEQ ID NO: 2)
C*G*U*ACGAUCUCGUAAACGCG + Synthego modified
EZ Scaffold

N*N*N* indicate 2′-O-methyl analogues and 3′-phosphorothioate internucleotide linkages

Per sample 1 μL Cas9 protein dilution (25 μM) and 5 μL sgRNA ‘targeting GFP’ or ‘non-targeting’ dilution (25 μM) were combined in a micro tube to form the RNP with a molar ratio of Cas9:gRNA=1:5. The contents were mixed by flicking the tubes, briefly centrifuged and incubated 10 min at room temperature.

For each sample 6 μL RNP were pipetted into a micro tube, 19 μL U2OS cell suspension with a density of 2 million/ml were added to reach a final concentration of 1 μM RNP, then kept on ice until the PM.

Cells were treated with 40 μm-4 μm and 60 μm-4 μm (L_{c}-W_{c}) devices at a pressure of 3 bar. Cell suspension only and cell suspension with ‘non-targeting’-RNP were used as controls. After the PM until seeding, cell suspension was kept at room temperature. All samples were centrifuged for 10 min at 100 g, resuspended in 100 μL of appropriate cell culture media, seeded in a 96 well cell culture plate and incubated at 37° C. 22 h later cells were trypsinised and transferred into a 24 well cell culture plate. 90 h after the PM cells were trypsinised and the expression of GFP was analysed with the cell analyser FACS Calibur™ (BD Biosciences) counting up to 5,000 total events.

In parallel, an aliquot of 15,000 cells at timepoint 22 h was seeded into a μ-slide 8 well microscopy chamber (Ibidi GmbH). 2 days later cells were incubated with DAPI (Sigma-Aldrich) 1 μg/ml in L-15 media for 20 min at 37° C. Cells were washed twice with PBS and covered with L-15 media. Live-microscopy was performed with Deltavision inverted microscope (DV Elite Imaging system, Olympus IX-71 inverted microscope, applied precision) equipped with 10× objective (UPLSAPO, 0.4 NA, WD 3.1 mm, Olympus).

### Determination of Cell Diameters

To determine cell diameters, cells were detached with Trypsin-EDTA (0.05%), trypsinization was stopped by addition of appropriate cell culture media. Afterwards the media was replaced with PBS and bright-field imaging was performed using Nikon Eclipse Ti (Nikon) equipped with 20×, 0.45 numerical aperture Plan Fluor air objective (Nikon). The cell diameter was determined using Fiji software²⁴.

### Determination of Cell Velocity

The mean cell velocity of HeLa K was calculated for each device and for each operating pressure (Table 1) by the analysis of the cell flow inside the channel using an inverted microscope (Zeiss, Axio Observer.A1 equipped with 5×, 0.12 numerical aperture A Plan air objective). Videos were recorded at 6000 fps (EoSens CL 1362, Mikrotron) and cell position was analysed by Fiji software²⁴. Mean cell velocity was calculated as distance travelled by cell along the channel divided by the correspondent time, calculated through the ratio between the number of frames and the recording frame rate (6000 fps).

TABLE 1

| | | | Reynolds | Fluid Shear | Mean cell |
|---|---|---|---|---|---|
| Device | Pressure | Flow rate | number, | Stress, τ | velocity |
| (L_{c}-W_{c}) | (bar) | (μL min⁻¹) | Re | (N m⁻²) | (mm s⁻¹) |
| 40 μm-6 μm | 3 | 531 | 8.9 | 194.5 | 496 |
| | 4 | 818 | 13.7 | 299.5 | 722 |
| | 5 | 1,170 | 19.6 | 428.5 | 1012 |
| 60 μm-6 μm | 3 | 482 | 8.1 | 176.4 | 437 |
| | 4 | 735 | 12.3 | 269.0 | 715 |
| | 5 | 1,021 | 17.1 | 374.0 | 1047 |
| 40 μm-4 μm | 3 | 448 | 7.5 | 164.2 | 514 |
| | 4 | 629 | 10.6 | 230.4 | 703 |
| | 5 | 928 | 15.6 | 339.8 | 998 |
| 60 μm-4 μm | 3 | 408 | 6.9 | 149.5 | 391 |
| | 4 | 600 | 10.1 | 219.5 | 670 |
| | 5 | 883 | 14.8 | 323.3 | 913 |

Table 1: Table of recorded and calculated flow parameters for four different devices (40 μm-6 μm; 60 μm-6 μm; 40 μm-4 μm; 60 μm-4 μm (L_{c}-W_{c})) for three operating pressure (3, 4 and 5 bar). Mean cell velocity for HeLa K cells was calculated by analysing the cell position in a microchannel over time.

### Statistical Analysis

Prism 6.0 (Graphpad) was used for statistical analysis. Kruskal-Wallis one-way analysis of variance was performed to test for significance of differences in a viability based on PI staining and in a fold increase in cell number. A p value higher than 0.05 was considered non-significant.

### Example 2: Device Operation by a Pressure and Flow Microfluidic Controller

A microfluidic system for the high-throughput processing of a small sample volumes under stable conditions was assembled. To ensure that the flow rate is not varying while the cells are processed, a pressure-based microfluidic controller (Fluigent MFCS™-EX) interfaced with a flow sensor and a commercial software (MAESFLO) to control pressure and measure flow rate in real time (FIG. 7) was used. The fluid pressure regulator is connected to the flow sensor via a chamber (vial (CMV)+tubing (CMT)) filled with the cell culture medium. To minimize the sample volume (about 20 μl) the cell suspension solution is loaded in a chamber (tubing (CST)) interconnecting the flow sensor and the microfluidic device.

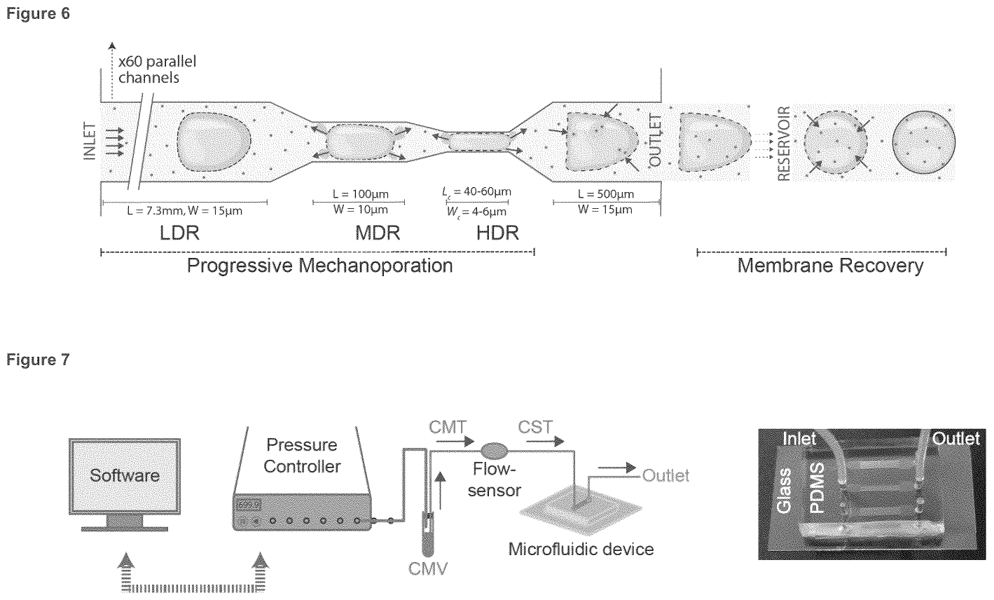

Subsequently, processed cells are collected through a chamber at the outlet of the microfluidic device (FIG. 7). The cell suspension solution flows from the microfluidic device inlet towards the microfluidic device outlet moving through 60 parallel channels (FIG. 14), where each channel is characterized by three regions that are able to act on cell deformation with different strength (FIG. 6): i) low deformation region (LDR): 15 μm wide, 7.3 mm long, 2) medium deformation region (MDR): 10 μm wide, 100 μm long, 3) high deformation region (HDR): 4 or 6 μm wide (W_{c}), 40 or 60 μm long (L_{c}), which is 550 μm far from the outlet chamber. Microfluidic devices are denoted by L_{c}_W_{c} based on their HDR dimensions. The compact device geometry allowed to integrate in a single chip three different devices (FIG. 7).

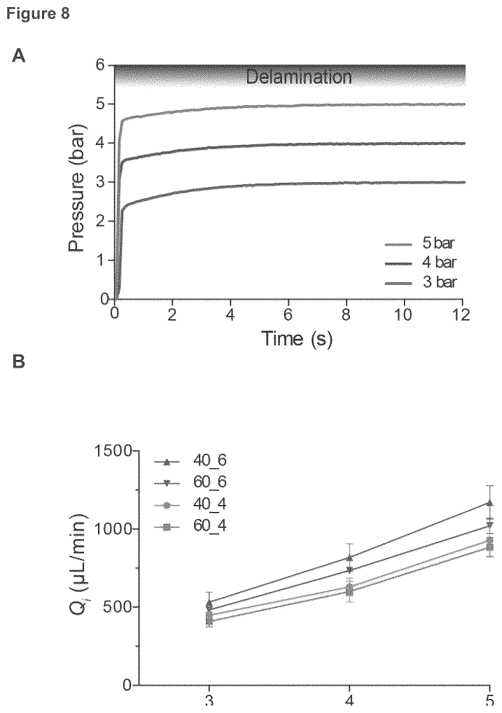

The flow inside the device was activated by changing the pressure inside the CMV and the corresponding flow rate was recorded. Previously, the flow inside the microfluidic chip has been controlled by either pressure-driven (constant pressure) or syringe-driven (constant flow) system, where the latter is limited by longer response and stabilization time and high fluctuations^{14,15}. Pressure-driven flow is preferable to process small sample volumes in a short time, but requires the employment of watertight, leakproof chip and a control of the flow rate to ensure process stability. Microfluidic chips made of different materials have been produced mostly employing microfabrication technologies on silicon or glass or PDMS. The latter allows for low cost, easy fabrication and rapid prototyping, facilitating fast adaptation of the device geometry²⁵. Here, the devices were produced by simple air plasma activated bonding of a microstructured PDMS element on a flat glass cover slip able to support high applied pressure without delamination. Optimal combination of punched holes (1 mm) in correspondence of the inlet chamber in PDMS element and tubing size (CST with O.D. 1.5 mm) guaranteed a watertight connection. The same results have been obtained before by using a second PDMS casting, making the fabrication process more complex and time consuming¹⁴. The device described herein is able to support pressure up to 5 bar (flow rate up to 1170 μL/min). Beyond 5.5 bar, delamination between the PDMS element and glass was observed. After 0.4 sec the controller is already able to provide 94% of the applied pressure (FIG. 8a). The flow rate was monitored in real time during the sample processing for three different applied pressures (3, 4 and 5 bar) and four different devices (FIG. 8b).

The Reynolds number (Re), the fluid shear stress (τ) and the average cell velocity for different devices and applied pressures were ranging from 6.9-19.6, 149.5-428.5 N m⁻² and 391-1047 mm s⁻¹, respectively (Table 1). The time for a cell to cross the entire length of the channel (8 mm) was 8-18 ms and the transition time in the MDR-HDR for an operating pressure of 3 bar was lower than 0.7 ms.

It was demonstrated that this system supports high pressure and is able to provide high flow rates with fast stabilization time, fundamental for processing small sample volume in a reproducible and reliable manner. Cells moving through the channel length experience a multistage deformation (FIG. 14) depending on the cell diameter/channel width ratio, cell mechanical properties and cell velocity. In particular, the cells are pre-deformed in the LDR/channel width comparable to the cell diameter) before their contact-based compression in the narrow constrictions (channels width smaller than the cell diameter). Membrane pores can already be formed in the LDR mostly due to hydrodynamic forces. Exactly, cells flowing in the LDR are subjected to high shear forces⁵ (up to 428.5 N m⁻²) and they are deformed under a flow regime dominated by inertial forces¹⁸ (Re>1). In the MDR-HDR cells undergo high mechanical compression that can enlarge and enrich pores. At the same time volume exchange under fast deformation^{16,17} (<0.7 ms) can be favoured through the opened pores (FIG. 6). The overall molecular uptake is the result of convective and diffusive transport of molecules from the cell surrounding to its interior through pores opened by hydrodynamic stress and contact-mediated cell compression. The resulting strain on the cells will be dependent not only on their size, but also on their mechanical properties, affecting both cell deformation²⁶ and membrane poration²⁷ and repair^{28,29}.

For different device geometry, the flow rate at the inlet, Qᵢ, varied with the device flow resistance, decreasing with the increase of flow resistance in each channel (R_{fc}), according to Hagen-Poiseuille equation, R_{fc}=12 μL/πwh³, where μ is the dynamic viscosity (dynes·s/cm²) and L, w and h are the length, width and height of the channel, respectively. In agreement with this relation, we observed a decrease in the flow rate with the increase of L_{c} and decrease in the W_{c}. For all four different devices dimensionless Reynolds number (Re), a ratio of inertial forces to viscous forces was calculated as, Re=4 Q_{c}/Pv, where Q_{c}=Qᵢ/60, is the volumetric flow rate (m³/s), P is the perimeter of LDR channel (m) and v is the kinematic viscosity (m²/s). The fluid shear stress (FSS, τ) for rectangular channel with h>w was calculated as, τ=6 μQ_{c}/hw², where h and w are the height and width of the LDR channel, respectively. (Table 1).

### Example 3: Analysis of Cell Size and Elasticity

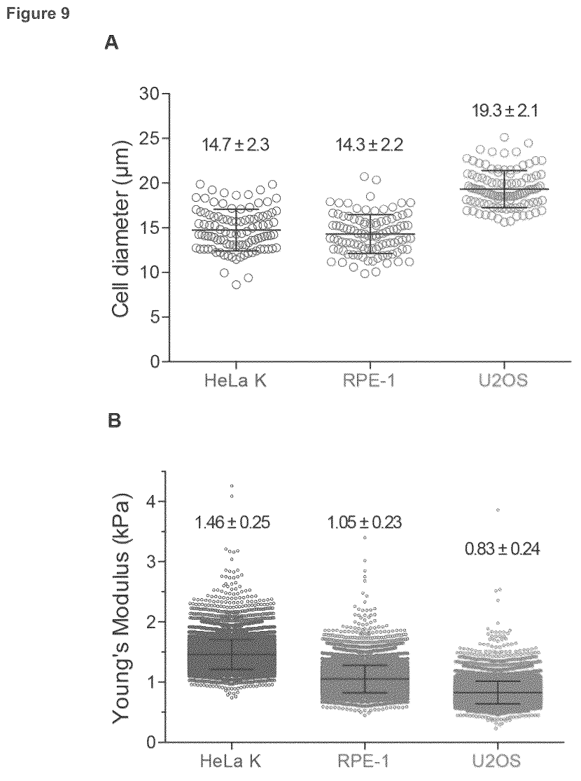

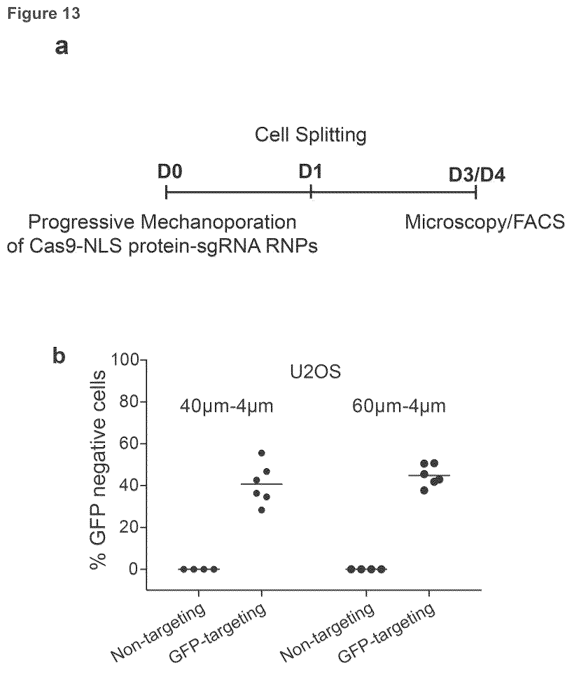

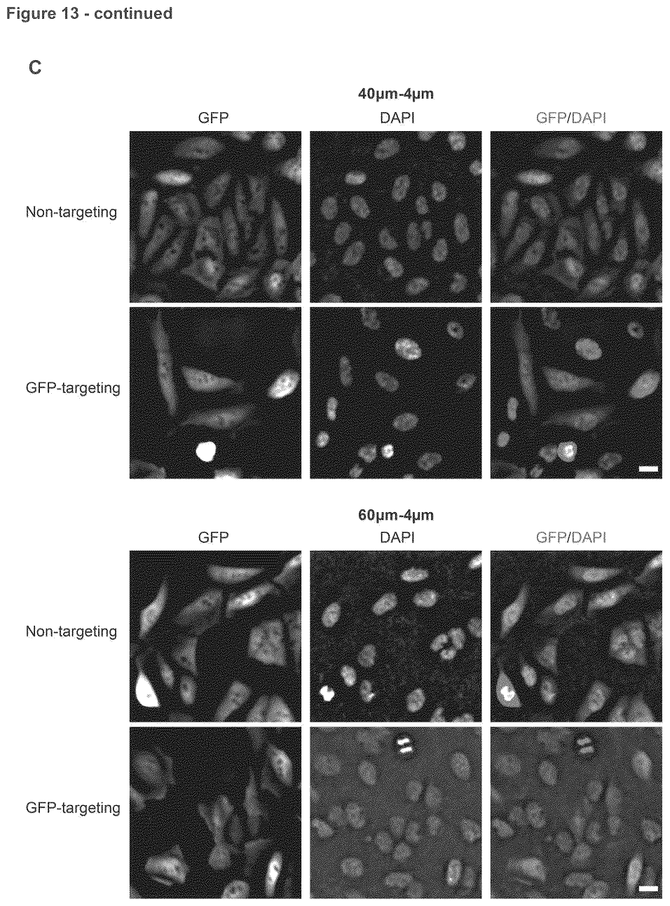

To assess the impact of cell mechanics on the extent of permeabilization, two different cell types were selected, Hela cells (Kyoto strain, HeLa K) and retina pigment epithelial cells (hTERT RPE-1). In addition, BJ fibroblasts and U2OS cells were included, respectively, for a direct comparison with a previous study¹⁴ and for characterizing a cellular model employed here to assess the applicability of PM in cell-based therapies (see FIG. 13). The size distribution of rounded cells was measured by bright-field image analysis determining their diameter after trypsin-mediated detachment (FIG. 9a). Cell elasticity was measured using real time deformability cytometry (RT-DC) (FIG. 9b), which relies on flowing cells through a microchannel where the hydrodynamic shear-induced deformation of a cell is analysed at throughput of 1000 cells/s in real time³⁰. Together, this revealed that RPE-1 and HeLa K cells have a similar diameter of (14.3±2.2 μm and 14.7±2.3 μm) but differ in elasticity (Young's modulus of 1.05±0.2 kPa versus 1.46±0.3 kPa, respectively). In contrast, the larger BJ fibroblasts and U2OS cells have similar size (19±2.6 μm and 19.3±2.1 μm) and elasticity (Young's modulus of 0.83±0.2 kPa for both) (FIG. 9).

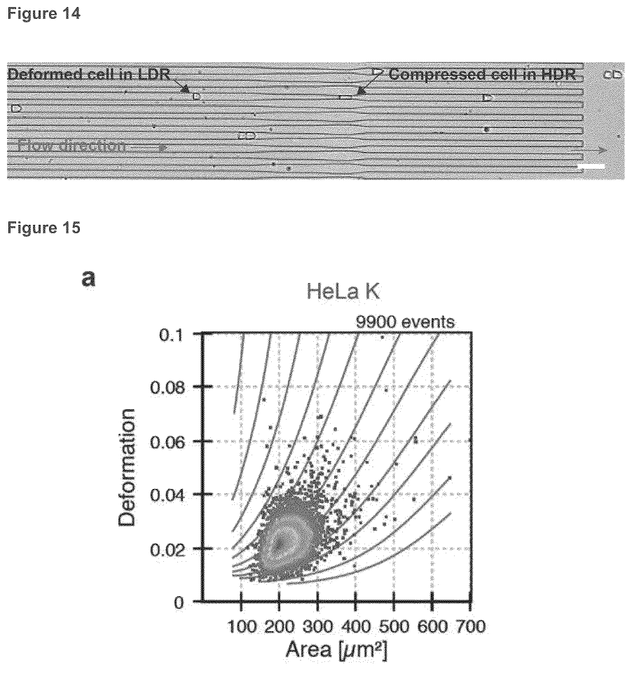

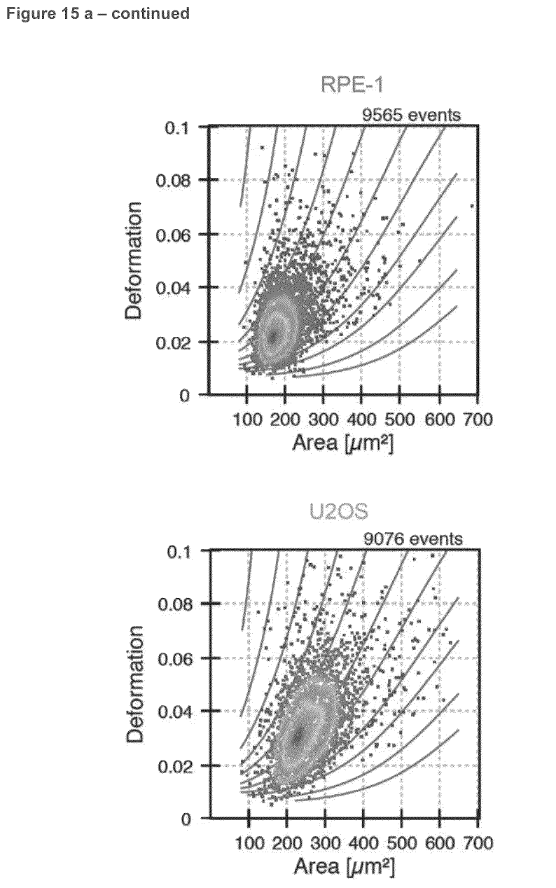

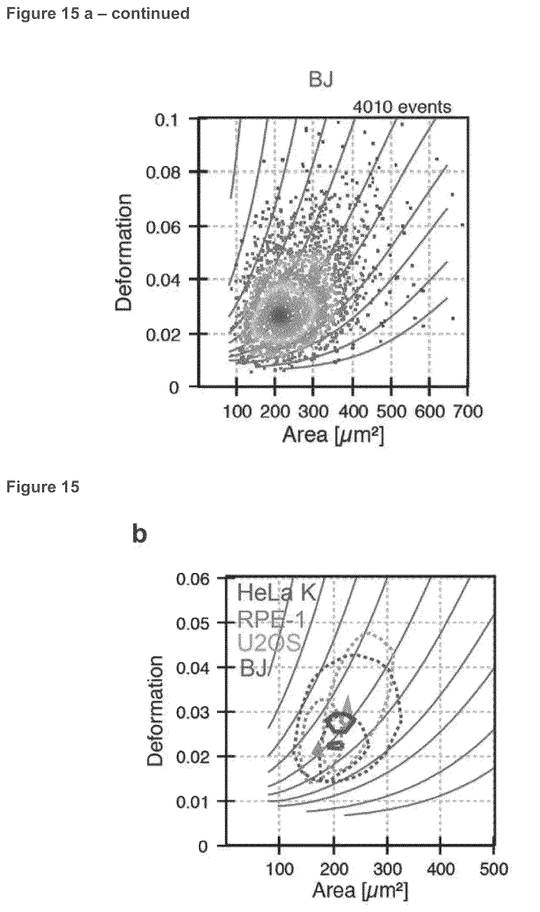

Delivery efficiency can vary with cell size, where larger cells can deform more in the three deformation regions, when travelling along the channel. In addition to the cell size, depending on their elasticity, cells deform differently (FIG. 15) which can lead to differences in the delivery efficiency. Thus, analysing RPE-1 and HeLa K (comparable size, different elasticity) allows to decouple these two parameters providing insights on the impact of cell elasticity on delivery efficiency.

### Example 4: Intracellular Delivery of Small Cargo Into Cells with Comparable Size and Different Elasticity

Biological studies and clinical applications often require the intracellular delivery of small impermeable cargo molecules, such as intracellular probes, dyes, inhibitors or peptides with molecular weight <5 kDa⁸. Therefore, the delivery efficiency of the PM system using small cargo, specifically 4 kDa Fitc-Dextran mimicking the size of above-mentioned molecules into different cell types was assessed.

Delivery of small cargo (4 kDa Fitc-Dextran) into Hela K and RPE-1 cells was analysed by Fluorescence Activated Cell Sorting (FACS) analysis. Two different devices, with same W_{c} of 6 μm and different L_{c} of 40 μm and 60 μm were used under two pressure conditions, 3 bar and 5 bar. The ratio between the cell diameter and constriction width was calculated and it ranges from 1.4 to 3.5 for both cell types. To gate for PM-specific uptake of Fitc-Dextran cells not flown through the device (Ctrl) or exposed to 4 kDa Fitc-Dextran without PM (Ctrl+Fitc-Dextran) in parallel (FIG. 16) were analysed.

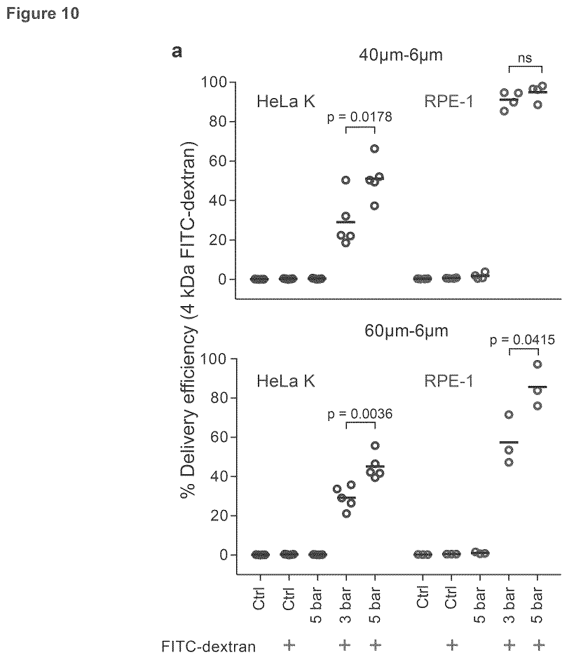

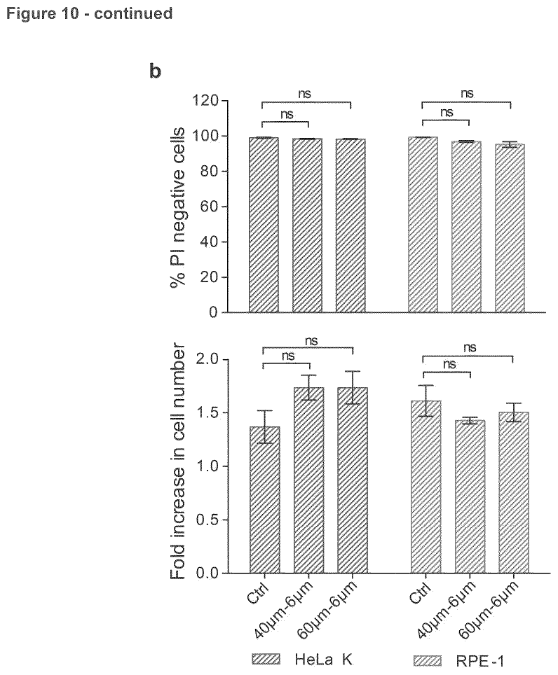

Delivery efficiency was calculated as the ratio between number of 4 kDa Fitc-Dextran positive cells and total number of analysed cells. While 40 μm-6 μm and 60 μm-6 μm (L_{c}-W_{c}) devices enabled the delivery of 4 kDa Fitc-Dextran into both, HeLa K and RPE-1 cells (FIG. 10a) differences in delivery efficiency were observed as a function of the operating pressure and cell type. Increasing the pressure (from 3 bar to 5 bar) increased the number of Fitc-positive HeLa K cells in both the devices 40 μm-6 μm (L_{c}-W_{c}) (from 29% to 51%) and 60 μm-6 μm (L_{c}-W_{c}) (from 29% to 45%), in agreement with a previously shown relation between pressure and delivery efficiency⁶. A similar trend was observed with RPE-1 cells in the 60 μm-6 μm (L_{c}-W_{c}) device (from 57% to 86%). In contrast, with the previously reported relation between L_{c} and delivery efficiency, the delivery efficiency was higher in the shorter device 40 μm-6 μm (up to 95%) than with longer device 60 μm-6 μm (from 57% to 86%), (FIG. 10a) for RPE-1 cells. Notably, for both devices, delivery efficiency was higher into RPE-1 that have a Young's Modulus lower than HeLa K cells, even though the diameter of both cell types is almost identical (FIG. 9a).

Effects of PM on cell viability were assessed by staining for dead cells with propidium iodide (PI) directly after the treatment with device using untreated cells as a negative control (Ctrl). Even at the highest applied pressure (5 bar), almost no dead cells were observed directly after treatment, neither in HeLa K nor in RPE-1 cells (FIG. 10b). Importantly, the proliferation rate of cells during 24 hours after the treatment was comparable to untreated control cells (FIG. 10b) suggesting that the mechanical stress cells experienced during PM had no longer term adverse effect on overall cell homeostasis.

It can be concluded that the PM delivers small cargo with up to 95% efficiency into RPE-1 cells independently of the applied pressure and without affecting cell viability and proliferation. For HeLa K cells, comparable in size but stiffer than RPE-1, delivery efficiency at a W_{c} of slightly less than half the cell diameter positively correlates with increasing pressure highlighting cell elasticity as an important intrinsic physical property for efficient mechanoporation.

### Example 5: Progressive Mechanoporation in Narrower Constriction Enhances Delivery Efficiency of Small Cargo Into Stiffer Cells

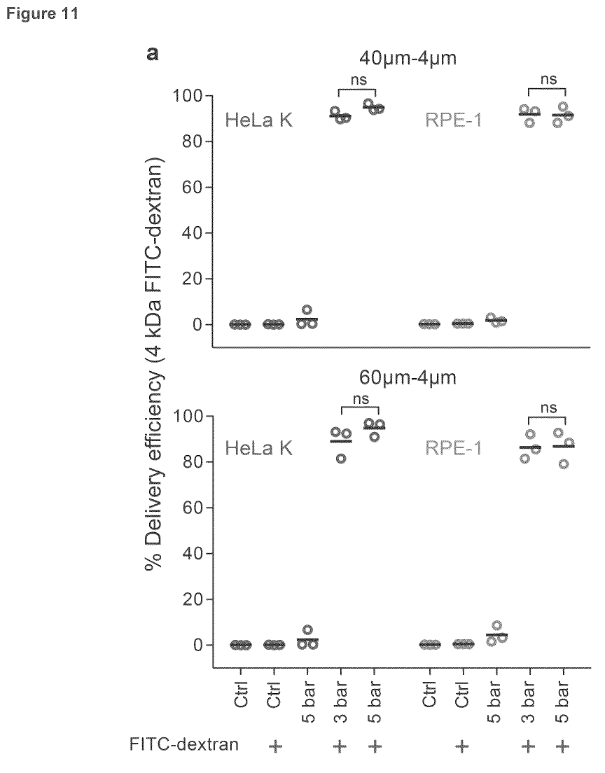

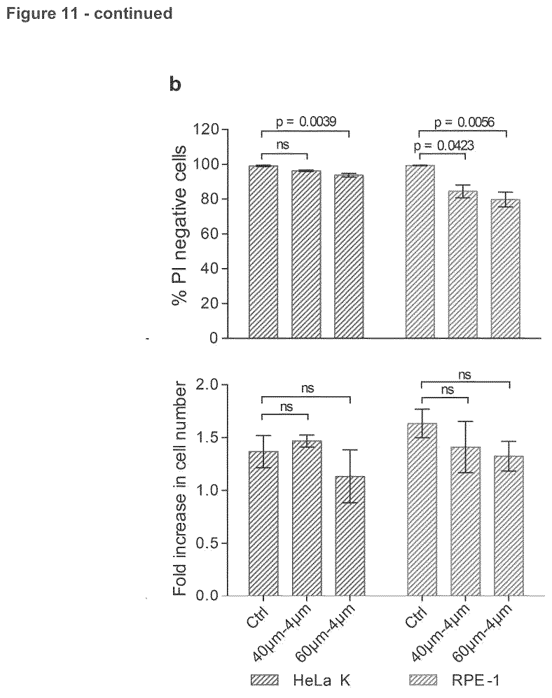

It was observed that at the same ratio of cell diameter to constriction width, delivery is more efficient into softer RPE-1 than into stiffer HeLa K cells (FIG. 10a). To increase the intracellular delivery into stiffer cells the contribution of progressive mechanoporation was enhanced by decreasing the W_{c} to 4 μm. Notably, the 40 μm-4 μm (L_{c}-W_{c}) device was able to provide higher delivery efficiency for Hela K (91%-96%) comparable to the one of RPE-1 (both 92%) for both the applied pressure (3 bar-5 bar), (FIG. 11a). This demonstrated that adapting device geometry is a strategy to efficiently deliver small cargos at lower pressure independent of the cell elasticity. For the longer constriction (60 μm-4 μm (L_{c}-W_{c})), a slightly lower delivery efficiency was measured, ranging from 89%-95% for Hela K and 86% -87% for RPE-1 cells (FIG. 11a). A pressure of 3 bar was sufficient to reach high intracellular delivery and high viability for HeLa K (>90%) and RPE-1 (>80%) cells and did not significantly affect proliferation capacity during the subsequent 24 hours. The longer device (60 μm-4 μm (L_{c}-W_{c})) decreased cell viability slightly directly after PM as reported previously⁶.

Thus, it can be concluded that the pressure of 3 bar combined with 4 μm W_{c} provided a perfect compromise between delivery efficiency, cell viability and proliferation capacity.

### Example 6: Intracellular Delivery of Bigger Molecules and Biologically Relevant Cargo

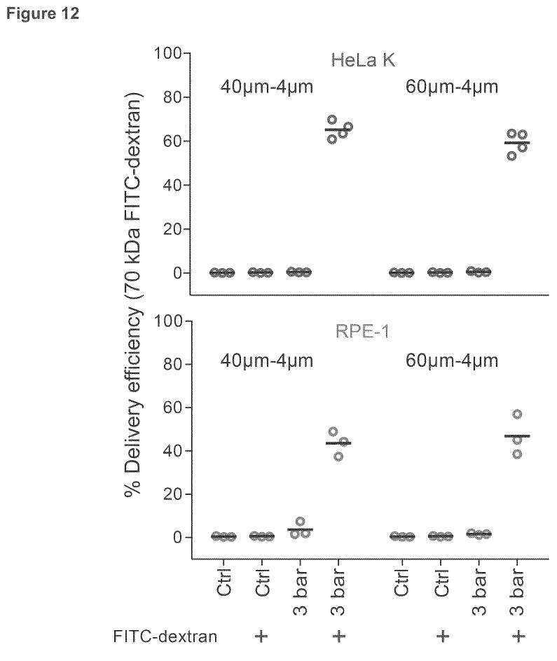

Applying an optimal constriction width (4 μm) and pressure (3 bar), the ability of PM to deliver larger cargo molecules that are similar to the size of therapeutically relevant antibodies, transcription factors or CRISPR/Cas9 gene-editing complexes was assessed. Firstly, the delivery of 70 kDa Fitc-Dextran was tested. A delivery efficiency of (65%, 59%) for HeLa K and (44%, 47%) for RPE-1 cells (FIG. 12) was obtained by using device with different L_{c} (40 μm, 60 μm). This demonstrated the ability of PM to deliver larger molecules albeit with reduced capability compared to the smaller ones.

To demonstrate the potential of PM for upcoming cell therapies based on gene-editing, a functional 190.8 kDa large Cas9 protein-single guide RNA ribonucleoprotein complex (Cas9-sgRNA RNP) was aimed to be delivered into cells. PM was used to deliver recombinant nuclear-localized Cas9 protein (Cas9-NLS) complexed with sgRNA targeting the green fluorescent protein (GFP) into an U2OS reporter cell line containing single copy of the GFP gene³¹. As a control, non-targeting sgRNA in complex with Cas9-NLS was employed. After four days, the percentage of GFP negative cells was analysed by FACS and fluorescence microscopy. Loss of GFP fluorescence is indicative of functional Cas9-gRNA delivery and subsequent knock out of the GFP gene (FIG. 13a). Indeed, delivery of Cas9-sgRNA RNPs targeting GFP but not a non-targeting control resulted in 41% and 45% GFP negative cells using 40 μm-4 μm and 60 μm-4 μm (L_{c}-W_{c}) devices, respectively (FIG. 13b, c).

In conclusion, a PDMS-based progressive mechanoporation system was developed enabling efficient and functional delivery of cargo molecules with high molecular weights highlighting its applicability for CRISPR/Cas9-mediated gene-editing.

### FURTHER REFERENCES

1 M. P. Stewart, A. Sharei, X. Ding, G. Sahay, R. Langer and K. F. Jensen, Nature, 2016, 538, 183-192.
2 M. P. Stewart, R. Langer and K. F. Jensen, Chemical Reviews, 2018, 118, 7409-7531.
3 A. Kumar, L. Mohan, P. Shinde, H.-Y. Chang, M. Nagai and T. S. Santra, in Handbook of Single Cell Technologies, Springer Singapore, 2018, pp. 1-29.
4 M. S. F. Clarke and P. L. McNeil, Journal of cell science, 1992, 102, 533-541.
5 D. M. Hallow, R. A. Seeger, P. P. Kamaev, G. R. Prado, M. C. LaPlaca and M. R. Prausnitz, Biotechnology and Bioengineering, 2008, 99, 846-854.
6 A. Sharei, J. Zoldan, A. Adamo, W. Y. Sim, N. Cho, E. Jackson, S. Mao, S. Schneider, M. J. Han, A. Lytton-Jean, P. A. Basto, S. Jhunjhunwala, J. Lee, D. A. Heller, J. W. Kang, G. C. Hartoularos, K. S. Kim, D. G. Anderson, R. Langer and K. F. Jensen, Proceedings of the National Academy of Sciences, 2013, 110, 2082-2087.
7 A. Sharei, R. Poceviciute, E. L. Jackson, N. Cho, S. Mao, G. C. Hartoularos, D. Y. Jang, S. Jhunjhunwala, A. Eyerman, T. Schoettle, R. Langer and K. F. Jensen, Integrative Biology (United Kingdom), 2014, 6, 470-475.
8 M. T. Saung, A. Sharei, V. A. Adalsteinsson, N. Cho, T. Kamath, C. Ruiz, J. Kirkpatrick, N. Patel, M. Mino-Kenudson, S. P. Thayer, R. Langer, K. F. Jensen, A. S. Liss and J. C. Love, Small, 2016, 12, 5873-5881.
9 T. DiTommaso, J. M. Cole, L. Cassereau, J. A. Buggé, J. L. Sikora Hanson, D. T. Bridgen, B. D. Stokes, S. M. Loughhead, B. A. Beutel, J. B. Gilbert, K. Nussbaum, A. Sorrentino, J. Toggweiler, T. Schmidt, G. Gyuelveszi, H. Bernstein and A. Sharei, Proceedings of the National Academy of Sciences, 2018, 115, E10907-E10914.
10 A. Klein, S. Hank, A. Raulf, E. F. Joest, F. Tissen, M. Heilemann, R. Wieneke and R. Tampé, Chemical Science, 2018, 9, 7835-7842.
11 J. Li, B. Wang, B. M. Juba, M. Vazquez, S. W. Kortum, B. S. Pierce, M. Pacheco, L. Roberts, J. W. Strohbach, L. H. Jones, E. Hett, A. Thorarensen, J. B. Telliez, A. Sharei, M. Bunnage and J. B. Gilbert, ACS Chemical Biology, 2017, 12, 2970-2974.
12 G. L. Szeto, D. Van Egeren, H. Worku, A. Sharei, B. Alejandro, C. Park, K. Frew, M. Brefo, S. Mao, M. Heimann, R. Langer, K. Jensen and D. J. Irvine, Scientific Reports, 2015, 5, 10276.
13 C. Tu, L. Santo, Y. Mishima, N. Raje, Z. Smilansky and J. Zoldan, Integrative Biology (United Kingdom), 2016, 8, 645-653.
14 K. H. Lam, A. Fernandez-Perez, D. W. Schmidtke and N. V. Munshi, Biomedical Microdevices, 2018, 20, 52.
15 W. Zeng, S. Li and Z. Wang, International Conference on Fluid Power and Mechatronics (FPM), Harbin, 2015, 711-715.
16 A. Liu, T. Yu, K. Young, N. Stone, S. Hanasoge, T. J. Kirby, V. Varadarajan, N. Colonna, J. Liu, A. Raj, J. Lammerding, A. Alexeev and T. Sulchek, Small, 2020, 16, 1903857.
17 A. Liu, M. Islam, N. Stone, V. Varadarajan, J. Jeong, S. Bowie, P. Qiu, E. K. Waller, A. Alexeev and T. Sulchek, Materials Today, 2018, 21, 703-712.
18 M. E. Kizer, Y. Deng, G. Kang, P. E. Mikael, X. Wang and A. J. Chung, Lab on a Chip, 2019, 19, 1747-1754.
19 M. P. Stewart, R. Langer and K. F. Jensen, Chemical Reviews, 2018, 118, 7409-7531.
20 C. A. Lino, J. C. Harper, J. P. Carney and J. A. Timlin, Drug Delivery, 2018, 25, 1234-1257.
21 B. H. Yip, Biomolecules, 2020, 10, 893.
22 S. Kim, D. Kim, S. W. Cho, J. Kim and J.-S. Kim. Genome Research, 24, 1012-1019.
23 T. Zerjatke, I. A. Gak, D. Kirova, M. Fuhrmann, K. Daniel, M. Gonciarz, D. Müller, I. Glauche and J. Mansfeld, Cell Reports, 2017, 19, 1953-1966.
24 J. Schindelin, I. Arganda-Carreras, E. Frise, V. Kaynig, M. Longair, T. Pietzsch, S. Preibisch, C. Rueden, S. Saalfeld, B. Schmid, J. Y. Tinevez, D. J. White, V. Hartenstein, K. Eliceiri, P. Tomancak and A. Cardona, Nature Methods, 2012, 9, 676-682.
25 A. Kumar and G. M. Whitesides, Applied Physics Letters, 1993, 63, 2002-2004.
26 A. Mietke, O. Otto, S. Girardo, P. Rosendahl, A. Taubenberger, S. Golfier, E. Ulbricht, S. Aland, J. Guck and E. Fischer-Friedrich, Biophysical Journal, 2015, 109, 2023-2036.
27 S. Modaresi, S. Pacelli, S. Subham, K. Dathathreya and A. Paul, Advanced Therapeutics, 2020, 3, 1900130.
28 P. L. McNeil and R. A. Steinhardt, Annual Review of Cell and Developmental Biology, 2003, 19, 697-731.
29 S. T. Cooper and P. L. McNeil, Physiological Reviews, 2015, 95, 1205-1240.
30 O. Otto, P. Rosendahl, A. Mietke, S. Golfier, C. Herold, D. Klaue, S. Girardo, S. Pagliara, A. Ekpenyong, A. Jacobi, M. Wobus, N. Töpfner, U. F. Keyser, J. Mansfeld, E. Fischer-Friedrich and J. Guck, Nature Methods, 2015, 12, 199-202.
31 J. Mircetic, I. Steinebrunner, L. Ding, J.-F. Fei, A. Bogdanova, D. Drechsel and F. Buchholz, Small Methods, 2017, 1, 4.
32 J M. Herbig, M. Krater, K. Plak, P. Müller, J. Guck and O. Otto, Flow Cytometry Protocols, Methods in Molecular Biology, Humana Press Inc., 2018, 1678, 347-369.
33 D. A. Braasch and D. R. Corey, Chemistry & biology, 2001, 8, 1-7.

1. A microfluidic device for introducing pores into and/or enhancing the diameter of pores in the cell membrane of a cell by cell deformation for delivery of cargo molecules into said cell, the device comprising:
an inlet and an outlet; and
at least one microfluidic channel positioned between said inlet and said outlet, defining a lumen, adapted to allow a cell and cargo molecules in a suspension solution to pass therethrough;
wherein the at least one microfluidic channel comprises at least two constrictions with different cross-sections, and
wherein one of said constrictions has a cross-section that is larger than the average cross-section of said cell and adapted to apply hydrodynamic forces to said cell and a second of said constrictions has a cross-section that is equal to or smaller than the average cross-section of said cell and adapted to apply contact-based compression forces to said cell, while allowing said cell to pass through said constrictions.

2. The microfluidic device of claim 1, wherein
the minimum dimension of the cross-section of the large constriction and/or the minimum dimension of the cross-section of the small constriction is between 2 μm and 20 μm; and/or wherein the area of the cross-section of the large constriction and/or the area of the cross-section of the small constriction is between 25 μm² and 1200 μm²; and/or wherein the large constriction and/or the small constriction extend over a length along the fluid flow direction between 10 μm and 50 mm; and/or wherein the distance between the large constriction and the small constriction along the fluid flow direction is between 50 μm and 5 mm; and/or wherein the minimum dimension of the cross-section of the microfluidic channel between the large and small constrictions is between 7 μm and 19 μm; and/or wherein the area of the cross-section of the microfluidic channel between the large and small constrictions is between 80 μm² and 1100 μm²;
and
wherein the ratio between the minimum dimension of the cross-section of the large constriction and the minimum dimension of the cross-section of the small constriction is between 1.2 and 7; and/or wherein the ratio between the area of the cross-section of the large constriction and the area of the cross-section of the small constriction is between 1.2 and 7; and/or wherein the ratio between the minimum dimension of the cross-section of the large constriction and the minimum dimension of the cross-section of the microfluidic channel between the large and small constrictions is between 1.1 and 3.5; and/or wherein the ratio between the area of the cross-section of the small constriction and the area of the cross-section of the microfluidic channel between the large and small constrictions is between 0.1 and 0.9.

3. The microfluidic device of claim 1, wherein
a. the ratio between the minimum dimension of the cross-section of the large constriction and the minimum dimension of the cross-section of the small constriction is between 1.2 and 7, preferably between 2.5 and 5, more preferably between 2.5 and 3.5; and/or
b. the ratio between the area of the cross-section of the large constriction and the area of the cross-section of the small constriction is between 1.2 and 7, preferably between 2.5 and 5, more preferably between 2.5 and 3.5; and/or
c. the ratio between the minimum dimension of the cross-section of the large constriction and the minimum dimension of the cross-section of the microfluidic channel between the large and small constrictions is between 1.1 and 3.5, preferably between 1.2 and 2.5, more preferably between 1.3 and 2.0; and/or
d. the ratio between the area of the cross-section of the small constriction and the area of the cross-section of the microfluidic channel between the large and small constrictions is between 0.1 and 0.9, preferably between 0.2 and 0.6, more preferably between 0.2 and 0.4.

4. The microfluidic device of claim 1, wherein said at least one microfluidic channel comprises three constrictions and the third of said three constrictions has a cross-section that is smaller than the average cross-section of said cell and adapted to apply contact-based compression forces to said cell while allowing said cell to pass through said constriction and wherein the cross-section of the third constriction is smaller than the cross-section of the second constriction.

5. The microfluidic device of claim 1, wherein the constrictions are arranged with descending cross-sections with regard to the flow direction of the cells; or wherein the constrictions are arranged with ascending cross-sections with regard to the flow direction of the cells.

6. A system comprising the microfluidic device of claim 1, and further comprising a fluid pressure regulator and/or a flow sensor which is connected to the inlet of the microfluidic device.

7. The system of claim 6, wherein the fluid pressure regulator regulates the fluid flow through the microfluidic channel, thereby applying a shear force on the cell for >1 ms at <1500 Pa, preferably for a minimum of 10 ms at 100-500 Pa, when passing the suspension solution through the large cell-deforming constriction.

8. The system of claim 6, further comprising an inlet chamber, which is in fluid communication with the inlet of the microfluidic device.

9. The system of claim 6, wherein a filter is positioned after the microfluidic device inlet.

10. A method for delivery of cargo molecules into a cell, the method comprising:
a. passing a suspension solution comprising said cell and said cargo molecules through the microfluidic device of any one of claims 1 to 5, wherein passing the suspension solution through the large cell-deforming constriction applies a shear force on the cell for >1 ms at <1500 Pa, preferably for a minimum of 10 ms at 100-500 Pa, whereby said cargo molecules are delivered into said cell; and
b. collecting cells into which said cargo molecules have been delivered.

11. The method of claim 10, wherein the cell has a Young's modulus of ≤3.5 kPa.

12. The method of claim 10, further comprising regulating the fluid flow through the microfluidic channel; and/or the flow rate through the microfluidic channel.

13. The method of claim 10, wherein passing the suspension solution through the small cell-deforming constriction applies a compression force and optionally a shear force on the cell for a maximum of 1 ms, wherein the shear force is 1500-6500 Pa.

14. The method of claim 10, wherein the ratio between the major and the minor axis of the cell is between 1.25 and 3.3 as it passes through the large cell-deforming constriction; and/or
wherein the ratio between the major and the minor axis of the cell is between 4 and 12.5 as it passes through the small cell-deforming constriction.

15. The method of claim 10, wherein the cell is incubated for a sufficient time to allow recovery of the cell membrane.

16. The method of claim 10, wherein the cell is a eukaryotic cell.

17. The method of claim 10, wherein the cargo molecules include small molecules, amino acids, proteins, nucleic acids, impermeable dyes, nano-carrier systems, CRISPR/Cas9 gene-editing complexes and combinations thereof.
